(19) United States
(12) Patent Application Publication
(10) Pub. No.: US 2014/0219972 A1
(43) Pub. Date: Aug. 7, 2014

(54) TISSUE ENGINEERING USING PROGENITOR CELLS TO CATALYZE TISSUE FORMATION BY PRIMARY CELLS

(71) Applicants: Janice Lai, Mountain View, CA (US); William Maloney, Menlo Park, CA (US); R. Lane Smith, Palo Alto, CA (US); Fan Yang, Stanford, CA (US)

(72) Inventors: Janice Lai, Mountain View, CA (US); William Maloney, Menlo Park, CA (US); R. Lane Smith, Palo Alto, CA (US); Fan Yang, Stanford, CA (US)

(73) Assignee: The Board of Trustees of the Leland Stanford Junior University, Palo Alto, CA (US)

(21) Appl. No.: 14/173,628

(22) Filed: Feb. 5, 2014

Related U.S. Application Data
(60) Provisional application No. 61/761,121, filed on Feb. 5, 2013.

(51) Int. Cl. A61K 35/12; A61K 35/32

(52) U.S. Cl. CPC A61K 35/35; A61K 35/32
USPC 424/93.7; 435/347; 435/373; 435/395

(57) ABSTRACT

Methods of regenerating tissue using progenitor cells in combination with primary cells from a target tissue are disclosed. In particular, progenitor cells catalyze proliferation and tissue production by primary cells allowing the use of fewer primary cells from a target tissue for effective tissue regeneration. Cell-based therapies combining progenitor cells and primary cells can be used for repair and regeneration of damaged tissue and organs for treating bodily injuries and degenerative diseases. For example, adipose-derived stem cells and neonatal articular chondrocytes, co-encapsulated in mixed or bilayered cultures in a hydrogel comprising chondroitin sulfate methacrylate and poly(ethylene)glycol diacrylate, generated cartilage that could be used for treatment of traumatic injuries or diseases involving cartilage degeneration. Moreover, the inventors showed that progenitor cells could be used to stimulate cartilage formation with a minimal number of primary cells, as few as 1% or less, in mixed cultures containing primary cells and progenitor cells.

### CROSS-REFERENCE TO RELATED APPLICATION

This application claims benefit under 35 U.S.C. §119(e) of provisional application 61/761,121, filed Feb. 5, 2013, which application is hereby incorporated by reference in its entirety.

### TECHNICAL FIELD

The present invention pertains generally to tissue engineering and regenerative medicine. In particular, the invention relates to methods of regenerating tissue using progenitor cells to catalyze proliferation and tissue production by primary cells.

### BACKGROUND

Cell-based therapy is a promising strategy for tissue repair and regeneration. In particular, primary cells, originating from the same tissue type as the damaged tissue in need of regeneration, possess the right phenotype for use in tissue replacement; however, the scarcity of available primary cells is a major hurdle preventing the widespread application of primary cell-based therapy for tissue repair. Furthermore, primary cells often cannot proliferate in vitro or they rapidly de-differentiate during expansion in vitro, further hindering their clinical application.

Cell-based approaches, for example, are sought after for cartilage repair and regeneration. Cartilage damage and loss is prevalent among adults and the older population, and can be caused by traumatic injury or degenerative diseases, such as arthritis. Due to its avascular nature, articular cartilage has limited self-repair potential (Mankin et al. (1982) J. Bone Joint Surg. Am. 64:460-466). Furthermore, the proliferation and regeneration potential of chondrocytes declines with age (Barbero et al. (2004) Osteoarthritis Cartilage 12:476-484). Damage to cartilage is often irreversible and if not treated properly, may alter mechanical loading and lead to the early onset of osteoarthritis (Griffin et al. (2005) Exerc. Sport Sci. Rev. 33:195-200).

Cell-based approaches using allogeneic neonatal chondrocytes offer a promising solution to cartilage regeneration. Neonatal chondrocytes, unlike other commonly used cell sources, such as autologous chondrocytes or mesenchymal stem cells from bone marrow, are highly proliferative, immune-privileged, and can readily produce abundant cartilage matrix, making neonatal chondrocytes a superior cell source for cartilage regeneration (Adkisson et al. (2001) Clin. Orthop. Relat. Res. 2001:S280-294; Adkisson et al. (2010) Stem Cell Res. 4:57-68; and Adkisson et al. (2010) Am. J. Sports Med. 38:1324-1333). However, the scarcity of neonatal chondrocytes and their rapid de-differentiation during expansion in vitro seriously hinders their clinical application.

There remains a need for improved cell-based therapies for repair and regeneration of damaged tissue and organs for treating bodily injuries and degenerative diseases.

### SUMMARY

The invention relates to cell-based therapies combining progenitor cells and primary cells for repair and regeneration of damaged tissue and organs for treating bodily injuries and degenerative diseases. In particular, progenitor cells are used to induce primary cells to proliferate and enhance tissue production by co-culture of the two cell-types in a three-dimensional scaffold. In a specific example, the inventors show that adipose-derived stem cells and neonatal articular chondrocytes, co-encapsulated in mixed or bilayered cultures in a hydrogel comprising chondroitin sulfate methacrylate (CS-MA) and poly(ethylene)glycol diacrylate (PEGDA), generated cartilage that could be used for treatment of traumatic injuries or diseases involving cartilage degeneration (see Example 1).

Thus, in one aspect, the invention includes a composition comprising a three-dimensional scaffold encapsulating progenitor cells and tissue-specific primary cells. The scaffold should be biocompatible with the encapsulated cells and allows production of the desired product from the primary cells. In one embodiment, the scaffold is a biomimetic scaffold that mimics certain aspects of the natural cell environment of the primary cell, such as the structure and function of the extracellular matrix (ECM). For example, the scaffold may be a hydrogel, which binds to paracrine signaling molecules released from the encapsulated cells. The progenitor cells and tissue-specific primary cells can be combined in the three-dimensional scaffold as a mixed culture, in which the progenitor cells and primary cells are uniformly mixed, or as a bilayered culture, in which the progenitor cells and primary cells are confined to separate layers. If combined as a mixed culture, the ratio of the two cell types can be adjusted to achieve optimum production of the desired cell product. The use of progenitor cells to catalyze tissue production by the primary cells allows a smaller number of primary cells to be used for tissue production than would be needed if the primary cells were used alone in tissue production. In one embodiment, the number of tissue-specific primary cells used in compositions for tissue production is the minimal number needed to promote a therapeutically effective amount of tissue production to treat a particular injury or disease involving tissue degeneration. In certain embodiments, one or more additional factors, such as nutrients, cytokines, growth factors, or antibiotics may be added to the scaffold to improve cell function or viability. The composition may also further comprise a pharmaceutically acceptable carrier.

In one embodiment, the invention includes a composition for generating new cartilage comprising adipose-derived stem cells and neonatal articular chondrocytes encapsulated in a hydrogel. In one embodiment, the hydrogel comprises chondroitin sulfate methacrylate (CS-MA) and poly(ethylene)glycol diacrylate (PEGDA). The neonatal articular chondrocytes, so encapsulated, produce cartilage in an amount effective for treatment of a subject in need of repair or replacement of cartilage. Thus, the composition can be used for treating a subject for a traumatic injury or a disease involving cartilage degeneration.

The adipose-derived stem cells and neonatal articular chondrocytes can be combined as a mixed culture or a bilayered culture in the hydrogel. In certain embodiments, adipose-derived stem cells and neonatal articular chondrocytes are combined in a mixed culture, wherein the ratio of adipose-derived stem cells to neonatal articular chondrocytes is about 25:75, about 50:50, about 75:25, about 90:10, about 95:5, about 99:1, or any ratio in between. In another embodiment, the percentage of neonatal articular chondrocytes in the mixed culture is 1% or less. In one embodiment, the number of neonatal articular chondrocytes is the minimal number needed to promote a therapeutically effective amount of cartilage production to treat an injury or disease involving cartilage degeneration.

In another aspect, the invention includes a method of treating a subject for tissue damage or loss, the method comprising administering a therapeutically effective amount of a composition comprising progenitor cells and tissue-specific primary cells, encapsulated in a three-dimensional scaffold, to the subject.

In one embodiment, the invention includes a method of treating a subject for cartilage damage or loss, the method comprising administering a therapeutically effective amount of a composition, as described herein, comprising adipose-derived stem cells and neonatal articular chondrocytes, to the subject.

In another aspect, the invention includes a method of generating new tissue in a subject, the method comprising administering a composition comprising progenitor cells and tissue-specific primary cells, encapsulated in a three-dimensional scaffold, to the subject.

In one embodiment, the invention includes a method of generating new cartilage in a subject, the method comprising administering a composition, as described herein, comprising adipose-derived stem cells and neonatal articular chondrocytes, to the subject.

In another aspect, the invention includes a method of preparing a composition for generating new cartilage in a subject, wherein the composition comprises a mixed culture of adipose-derived stem cells and neonatal articular chondrocytes. The method comprises: a) mixing chondroitin sulfate methacrylate (CS-MA) and poly(ethylene)glycol diacrylate (PEGDA) with water; b) adding adipose-derived stem cells and neonatal articular chondrocytes and media suitable for growth of the adipose-derived stem cells and neonatal articular chondrocytes to form a suspension; and c) inducing crosslinking of the PEGDA and CSMA to form a hydrogel. In certain embodiments, the method further comprises culturing the adipose-derived stem cells and neonatal articular chondrocytes in the hydrogel under conditions in which the cells proliferate before implantation of the composition in a subject.

In another aspect, the invention includes a method of preparing a composition for generating new cartilage in a subject, wherein the composition comprises a bilayered culture of adipose-derived stem cells and neonatal articular chondrocytes. The method comprises: a) preparing a first hydrogel encapsulating adipose-derived stem cells; b) preparing a second hydrogel encapsulating neonatal articular chondrocytes; and combining the two hydrogels into a bilayered hydrogel by bringing the first hydrogel and the second hydrogel in contact with each other. In one embodiment, the first hydrogel and the second hydrogel comprise PEGDA and CSMA. In certain embodiments, the method further comprises culturing the adipose-derived stem cells and neonatal articular chondrocytes in the hydrogel under conditions in which the cells proliferate before implantation of the composition in a subject.

In another aspect, the invention includes a composition comprising a hydrogel encapsulating neonatal articular chondrocytes and conditioned medium, wherein the medium has been conditioned by adipose-derived stem cells. In one embodiment, the hydrogel comprises PEGDA and CSMA. In certain embodiments, the method further comprises culturing the neonatal articular chondrocytes in the hydrogel under conditions in which the cells proliferate before implantation of the composition in a subject.

In another aspect, the invention includes a method of preparing a composition for generating new cartilage in a subject, wherein the composition comprises neonatal articular chondrocytes and conditioned medium. The method comprises: a) mixing chondroitin sulfate methacrylate (CS-MA) and poly(ethylene)glycol diacrylate (PEGDA) with water; b) adding neonatal articular chondrocytes, media conditioned by adipose-derived stem cells, and media suitable for growth of the neonatal articular chondrocytes to form a suspension; and c) inducing crosslinking of the PEGDA and CSMA to form the hydrogel.

The compositions described herein may be administered by any suitable method, such as by injection or implantation locally into an area of tissue damage or loss. For example, compositions, described herein, for treatment of cartilage loss or damage may be administered by injection or implantation locally into an area of cartilage damage or loss, such as a damaged joint of a subject.

In another aspect, the invention includes a kit comprising a composition for generating new tissue, as described herein, or reagents and cells for preparing such a composition (e.g., reagents for preparing a three-dimensional scaffold, progenitor cells, primary cells, media, and optionally one or more other factors, such as growth factors, ECM components, antibiotics, and the like). The kit may also comprise means for delivering the composition to a subject and instructions for treating a traumatic injury or a disease involving tissue degeneration.

In one embodiment, the invention includes a kit comprising a hydrogel composition for generating new cartilage, as described herein, or reagents and cells for preparing such a composition (e.g., chondroitin sulfate methacrylate (CS-MA), poly(ethylene)glycol diacrylate (PEGDA), adipose-derived stem cells, neonatal articular chondrocytes, media, and optionally one or more other factors, such as growth factors, ECM components, antibiotics, and the like). The kit may also comprise means for delivering the composition to a subject and instructions for treating a traumatic injury or a disease involving cartilage degeneration.

These and other embodiments of the subject invention will readily occur to those of skill in the art in view of the disclosure herein.

### BRIEF DESCRIPTION OF THE FIGURES

FIGS. 1A-1E show a schematic representation of the experimental design. In order to examine the interaction between adipose-derived stem cells (ADSCS) and neonatal chondrocytes (CHONS), three different in vitro culture models were used: conditioned medium (FIG. 1A)—cells were cultured with supplementation of conditioned medium from the other cell type (CM), bi-layer (FIG. 1B)—culture confined the two cell types to separate layers with no direct cell-cell contact, but allowing paracrine signals to diffuse into the adjacent layer, and mixed cell (FIG. 1C)—mixed cultures of the two cell types in one scaffold at different cell ratios. By changing the ratio of the two cell types in a 3D volume, we can tune the spatial distribution and distance between a chondrocyte from an ADSC, thereby changing the paracrine signal concentration they are sensing (FIG. 1D). Human adult ADSCS and bovine neonatal CHONS were encapsulated in 3D biomimetic hydrogels and cultured in vitro for 21 days in chondrogenic medium supplemented with TGF-β3. FIG. 1E shows that in the mixed cell culture, increasing ADSC ratio while keeping the overall cell density constant at 15 million/ml leads to a linear increase in the number of ADSCS that are within effective communication distance (250 μm) of a CHON.

FIGS. 2A-2F show the gene expression of encapsulated cells in the three different types of co-culture models as illustrated in FIGS. 1A-1C, including CM: conditioned medium, Bi: bi-layered, and mixed co-culture at various ratios ranging from 75C:25A to 10C: 90A (C: chondrocytes, A: ADSCS). To distinguish the fate of each cell type, specie-specific primers were used to identify the gene expression of human ADSCS and bovine CHONS in the xenogenic culture. Human-specific (FIGS. 2A-2C) and bovine-specific (FIGS. 2D-2F) gene expression were compared at day 21 relative to day 1 ADSC and bAC controls. Conditioned medium treatment and bi-layered co-culture led to minimal changes in cartilage marker expression, including Aggrecan (Agg) and type II collagen (COL2). In contrast, all groups using mixed co-culture at all ratios led to about 6-fold higher expression of Agg and about 20-fold higher expression in COL2 by human ADSCS. Meanwhile, mixed co-culture also led to markedly decreased undesirable expression of the fibrocartilage marker, type I collagen (COLI) compared with the human ADSCS cultured alone (control). For bovine chondrocytes, mixed co-culture led to a maintained cartilage phenotype and slightly decreased expression of the fibrocartilage marker COL1. Conditioned medium and bi-layered co-culture led to a slight decrease in Agg and COL2 expression in chondrocytes.

FIGS. 3A-3G show biochemical analyses of cell proliferation, matrix production and mechanical properties of the cell-laden scaffolds by the end of the 21-day culture. Only mixed co-culture at various ratios, but not CM or bi-layered culture, led to markedly enhanced cell proliferation and cartilage matrix production. FIG. 3A shows measurements of DNA content at day 1 and 21, which were used to evaluate cell proliferation over time. In the ADSC control group, DNA content at day 21 was reduced to 29% of day 1 DNA content (FIG. 3A). Both conditioned medium and bi-layer co-cultures had significantly higher numbers of ADSCS than that of ADSC control at day 21. To quantify cartilage matrix production, sulfated glycosaminioglycan (sGAG) content (FIG. 3B) and total collagen content (FIG. 3C) were measured at day 21. SGAG and collagen per wet weight exhibited similar trends. FIG. 3D shows compressive moduli of the cell-laden samples by the end of 21 days in culture. To compare the extent of cell number and matrix production changes as a result of variation in cell ratio, the interaction index, which is the measured matrix content normalized by the expected matrix content based on the matrix content was measured in the CHON and ADSC control groups. The interaction index for DNA/w.w. (FIG. 3E), GAG/w.w. (FIG. 3F), and collagen/w.w. (FIG. 3G) increased with an increase in ADSC ratio in the mixed cell culture. FIGS. 3E-3G show the effects of cell ratio variation on cell proliferation and cartilage matrix production. The measured DNA (FIG. 3E), sGAG (FIG. 3F), and collagen (FIG. 3G) were compared against expected values. At each cell ratio, the interaction index, which is the measured matrix content (DNA, sGAG, or collagen) in the mixed co-culture group normalized by the expected matrix content, based on the measured matrix content in the CHON and hADSC alone groups, was calculated. The interaction for DNA, sGAG, and collagen per wet weight in all the mixed co-culture groups were higher than 1.

FIGS. 4A-4F show type II collagen (COL2) immunostaining in the three cell co-culture models. The differential effects of conditioned medium (CM), bi-layer (Bi), and mixed cell culture on cartilage matrix production were evident in the spatial organization of neo-cartilage within the 3D hydrogels as shown by the type II collagen immunostaining (FIG. 4A). Conditioned medium and bi-layer cultures did not show obvious changes in type II collagen production for either cell type. In contrast, mixed co-culture with all ratios led to formation of neo-cartilage nodules within the 3D hydrogels, with increasing size of each nodule as the ratio of ADSCS increased (FIG. 4A). To determine the distribution of the two cell types in the mixed cell cultures, ADSCS were membrane-labeled (red) prior to encapsulation in the hydrogels; FIG. 4B shows co-localization of type II collagen (top row) with labeled ADSCS (middle row) along with DAPI nuclei staining (bottom row). It was revealed that ADSCs were not present in cartilage nodules. Scale bars, 100 μm. FIGS. 4C-4E show the quantification of type II collagen immunostaining images, including cartilage nodule size at different ratios of ADSC at day 7 (FIG. 4C), day 14 (FIG. 4D), and day 21 (FIG. 4E), as well as the total percentage of area occupied by cartilage nodules at different cell ratios at days 7, 14, and 21 (FIG. 4F). Both the cartilage nodule size as well as the total area of hydrogel being replaced by cartilage nodules increased with an increase in ADSC ratio in the mixed cell culture.

FIGS. 5A-5D show histograms showing the distribution of intercellular distances between ADSCS and CHONS that are within effective communication distance (250 μm) from a CHON in a mixed cell culture with (FIG. 5A) 25% ADSC, (FIG. 5B) 50% ADSC, (FIG. 5C) 75% ADSC, and (FIG. 5D) 90% ADSC.

FIG. 6 shows Agg, COL1, and COL2 expression in human ADSCS and bovine CHONS at day 1 and day 21 when cultured alone.

FIG. 7 shows the gross appearance of freeze-dried cell hydrogel constructs at day 1 and day 21 (scalebar=10 mm).

FIG. 8 shows immunostaining of type II collagen in conditioned medium, bi-layer, and mixed cell culture groups at days 7 (top row) and 14 (bottom row). Cells were evenly distributed in the hydrogel construct at day 7. At day 14, cell aggregates and cartilage nodules (type II collagen positive) were observed in all the mixed cell culture groups (scale bars=100 μm).

FIG. 9 shows immunostaining of type I collagen in conditioned medium, bi-layer, and mixed cell cultures at day 21. Type I collagen was stained minimally.

### DETAILED DESCRIPTION

The practice of the present invention will employ, unless otherwise indicated, conventional methods of medicine, biology, biomaterials science, pharmacology, chemistry, biochemistry, recombinant DNA techniques and immunology, within the skill of the art. Such techniques are explained fully in the literature. See, e.g., G. Vunjak-Novakovic and R. I. Freshney Culture of Cells for Tissue Engineering (Wiley-Liss, 1^{st} edition, 2006); Biomaterials Science: An Introduction to Materials in Medicine (B. D. Ratner, A. S. Hoffman, F. J. Schoen, J. E. Lemons eds., Academic Press, 2^{nd} edition, 2004); An Introduction to Biomaterials (Biomedical Engineering, J. O. Hollinger ed., CRC Press, 2^{nd} edition, 2011); Biomaterials Science: An Integrated Clinical and Engineering Approach (Y. Rosen and N. Elman eds., CRC Press, 1^{st} edition, 2012); Arthritis Research: Methods and Protocols, Vols. 1 and 2: (Methods in Molecular Medicine, Cope ed., Humana Press, 2007); Cartilage and Osteoarthritis (Methods in Molecular Medicine, M. Sabatini P. Pastoureau, and F. De Ceuninck eds., Humana Press; 2004); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); and Sambrook et al., Molecular Cloning: A Laboratory Manual (3^{rd} Edition, 2001).

All publications, patents and patent applications cited herein, whether supra or infra, are hereby incorporated by reference in their entireties.

I. Definitions

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “a cell” includes a mixture of two or more cells, and the like.

The term “about,” particularly in reference to a given quantity, is meant to encompass deviations of plus or minus five percent.

As used herein, the term “conditioned medium” refers to a medium in which a specific cell or population of cells has been cultured, and then removed. When cells are cultured in a medium, they may secrete cellular factors that can provide trophic support to other cells. Such trophic factors include, but are not limited to hormones, cytokines, extracellular matrix (ECM), proteins, vesicles, antibodies, and granules. The medium containing the cellular factors is the conditioned medium.

“Biocompatible” generally refers to a material and any metabolites or degradation products thereof that are generally non-toxic to the recipient and do not cause any significant adverse effects to the subject.

“Hydrogel” refers to a substance formed when an organic polymer (natural or synthetic) is cross-linked via covalent, ionic, or hydrogen bonds to create a three-dimensional open-lattice structure which entraps water molecules to form a gel. Biocompatible hydrogel refers to a polymer that forms a gel which is not toxic to living cells, and allows sufficient diffusion of oxygen and nutrients to the entrapped cells to maintain viability.

“Mammalian cell” refers to any cell derived from a mammalian subject suitable for transplantation into the same or a different subject. The cell may be xenogeneic, autologous, or allogeneic. The cell can be a primary cell obtained directly from a mammalian subject. The cell may also be a cell derived from the culture and expansion of a cell obtained from a subject. For example, the cell may be a stem cell. Immortalized cells are also included within this definition. In some embodiments, the cell has been genetically engineered to express a recombinant protein and/or nucleic acid.

The term “progenitor cell” refers to a cell which is capable of differentiating into a specific type of cell. Progenitor cells include, but are not limited to, progenitor cells from various types of tissues, such as mesenchymal stromal cells from bone marrow, endothelial progenitor cells, muscle progenitor cells (e.g., satellite cells), pancreatic progenitor cells, periosteum progenitor cells, neural progenitor cells, blast cells, intermediate progenitor cells, and stem cells, including stem cells from embryos, umbilical cord, or adult tissues, or induced pluripotent stem cells.

The term “stem cell” refers to a cell that retains the ability to renew itself through mitotic cell division and that can differentiate into a diverse range of specialized cell types. Mammalian stem cells can be divided into three broad categories: embryonic stem cells, which are derived from blastocysts, adult stem cells, which are found in adult tissues, and cord blood stem cells, which are found in the umbilical cord. In a developing embryo, stem cells can differentiate into all of the specialized embryonic tissues. In adult organisms, stem cells and progenitor cells act as a repair system for the body by replenishing specialized cells. Totipotent stem cells are produced from the fusion of an egg and sperm cell. Cells produced by the first few divisions of the fertilized egg are also totipotent. These cells can differentiate into embryonic and extraembryonic cell types. Pluripotent stem cells are the descendants of totipotent cells and can differentiate into cells derived from any of the three germ layers. Multipotent stem cells can produce only cells of a closely related family of cells (e.g., hematopoietic stem cells differentiate into red blood cells, white blood cells, platelets, etc.). Unipotent cells can produce only one cell type, but have the property of self-renewal, which distinguishes them from non-stem cells.

As used herein, the term “cell viability” refers to a measure of the amount of cells that are living or dead, based on a total cell sample. High cell viability, as defined herein, refers to a cell population in which greater than 85% of all cells are viable, preferably greater than 90-95%, and more preferably a population characterized by high cell viability containing more than 99% viable cells.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

“Transplant” refers to the transfer of a cell, tissue, or organ to a subject from another source. The term is not limited to a particular mode of transfer. Encapsulated cells may be transplanted by any suitable method, such as by injection or surgical implantation.

The term “arthritis” includes, but is not limited to, osteoarthritis, rheumatoid arthritis, lupus-associated arthritis, juvenile idiopathic arthritis, reactive arthritis, enteropathic arthritis and psoriatic arthritis.

The term “disease involving cartilage degeneration” is any disease or disorder involving cartilage and/or joint degeneration. The term “disease involving cartilage degeneration” includes disorders, syndromes, diseases, and injuries that affect spinal discs or joints (e.g., articular joints) in animals, including humans, and includes, but is not limited to, arthritis, chondrophasia, spondyloarthropathy, ankylosing spondylitis, lupus erythematosus, relapsing polychondritis, and Sjogren's syndrome.

By “therapeutically effective dose or amount” of a composition comprising progenitor cells and tissue-specific primary cells or a composition comprising primary cells and conditioned media from a culture comprising progenitor cells is intended an amount that, when administered as described herein, brings about a positive therapeutic response in a subject having tissue damage or loss, such as an amount that results in the generation of new tissue at a treatment site. The exact amount required will vary from subject to subject, depending on the species, age, and general condition of the subject, the severity of the condition being treated, mode of administration, and the like. An appropriate “effective” amount in any individual case may be determined by one of ordinary skill in the art using routine experimentation, based upon the information provided herein.

For example, a therapeutically effective dose or amount of a composition comprising adipose-derived stem cells and neonatal articular chondrocytes or a composition comprising neonatal articular chondrocytes and conditioned media from a culture comprising adipose-derived stem cells is intended an amount that, when administered as described herein, brings about a positive therapeutic response in a subject having cartilage damage or loss, such as an amount that results in the generation of new cartilage at a treatment site (e.g., a damaged joint). For example, a therapeutically effective dose or amount could be used to treat cartilage damage or loss resulting from a traumatic injury or a degenerative disease, such as arthritis or other disease involving cartilage degeneration. Preferably, a therapeutically effective amount restores function and/or relieves pain and inflammation associated with cartilage damage or loss.

The terms “subject,” “individual,” and “patient,” are used interchangeably herein and refer to any mammalian subject for whom treatment or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, and hamsters; and primates.

II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention relates to methods of regenerating tissue using progenitor cells in combination with primary cells from a target tissue. In particular, progenitor cells catalyze proliferation and tissue production by primary cells allowing the use of fewer primary cells from a target tissue for effective tissue regeneration. The use of progenitor cells in combination with primary cells is highly advantageous given the scarcity of available primary cells, the inability of many primary cells to proliferate, and their tendancy to rapidly de-differentiate when cultured by themselves in vitro. Cell-based therapies combining progenitor cells and primary cells can be used for repair and regeneration of damaged tissue and organs for treating bodily injuries and degenerative diseases.

In a specific example, the inventors have shown that adipose-derived stem cells and neonatal articular chondrocytes, co-encapsulated in mixed or bilayered cultures in a hydrogel comprising chondroitin sulfate methacrylate (CS-MA) and poly(ethylene)glycol diacrylate (PEGDA), generated cartilage that could be used for treatment of traumatic injuries or diseases involving cartilage degeneration (see Example 1). The hydrogel served as a three-dimensional scaffold controlling intercellular distance between the progenitor cells and primary cells. The hydrogel also retained released paracrine signaling molecules allowing paracrine signal distribution to the primary cells. Three co-culture models were tested: a mixed culture of primary cells and progenitor cells, a bilayered culture with primary cells and progenitor cells in separate layers, and a culture of primary cells with conditioned media from progenitor cells. Of the three co-culture models tested, the mixed culture model provided the greatest degree of paracrine signal distribution to the primary cells, as well as intercellular contact between the primary cells and progenitor cells, and also the highest level of cartilage formation. Moreover, the inventors showed that progenitor cells could be used to stimulate cartilage formation with a minimal number of primary cells, as few as 1% or less, in mixed cultures containing primary cells and progenitor cells. Most unexpectedly, larger cartilage nodules formed as the number of chondrocytes was decreased in mixed cultures. Thus, a minimal number of primary cells in combination with progenitor cells can be used to achieve effective tissue repair.

In order to further an understanding of the invention, a more detailed discussion is provided below regarding cell-based therapies using progenitor cells in combination with tissue-specific primary cells.

The compositions for regenerating, replacing, or repairing tissue comprise a three-dimensional scaffold encapsulating progenitor cells and tissue-specific primary cells. The progenitor cells and tissue-specific primary cells can be combined in the scaffold as a mixed culture, in which the progenitor cells and primary cells are uniformly mixed, or as a bilayered culture, in which the progenitor cells and primary cells are confined to separate layers. If combined as a mixed culture, the ratio of the two cell types can be adjusted to achieve optimum production of the desired cell product. The three dimensional scaffold can be used to control the intercellular distance between the progenitor cells and primary cells and may bind and retain released paracrine signaling molecules allowing paracrine signal distribution to the primary cells.

Any three-dimensional scaffold that is biocompatible with the encapsulated cells and that allows production of the desired product from the primary cells may be used. Suitable biocompatible hydrogels for cell encapsulation are known and include, but are not limited to, hydrogels comprising polysaccharides, polyphosphazenes, poly(acrylic acids), poly(methacrylic acids), copolymers of acrylic acid and methacrylic acid, poly(alkylene oxides), poly(vinyl acetate), polyvinylpyrrolidone (PVP), and copolymers and blends of each. Polysaccharides that can be used include alginate, chitosan, hyaluronan, and chondroitin sulfate. See, e.g., Lee et al. (2008) Tissue Eng. Part A. 14(11):1843-1851; Hwang et al. (2007) Methods Mol. Biol. 407:351-373; Hwang et al. (2006) Stem Cells 24, 284-291; Lu et al. (2013) Int. J. Nanomedicine 8:337-350; Peng et al. (2012) Nanotechnology 23(48):485102; Pok et al. (2013) Acta Biomater. 9(3):5630-5642; Phadke et al. (2013) Eur. Cell Mater. 25:114-129; herein incorporated by reference.

In order to improve cell viability and tissue production, a biomimetic scaffold can be used that mimics certain aspects of the natural cell environment of the primary cell, such as the structure and function of the extracellular matrix (ECM). For example, a scaffold containing one or more ECM components can be used, such as a composite hydrogel scaffold containing at least one ECM component selected from the group consisting of a proteoglycan (e.g., chondroitin sulfate, heparan sulfate, and keratan sulfate), a non-proteoglycan polysaccharide (e.g., hyaluronic acid), a fiber (e.g., collagen and elastin), and any other ECM component (e.g., fibronectin and laminin). Preferably, the scaffold binds to one or more paracrine signaling molecules released from the encapsulated cells.

Chondroitin sulfate is one of the predominant structural proteoglycans in many tissues, including skin, cartilage, tendons, and heart valves and, therefore, is useful to include in biomimetic scaffolds for many tissue engineering applications. Hydrogels containing chondroitin sulfate can be prepared by modifying chondroitin sulfate with methacrylate groups followed by photopolymerization. The hydrogel properties can be readily controlled by the degree of methacrylate substitution and macromer concentration in solution prior to polymerization. Copolymer hydrogels of chondroitin sulfate and an inert polymer, such as polyethylene glycol (PEG) or polyvinyl alcohol (PVA) may also be used. See, e.g., Varghese et al. (2008) Matrix Biol. 27(1):12-21; Strehin et al. (2010) Biomaterials. 31(10):2788-2797; herein incorporated by reference.

The primary cells chosen for encapsulation depend on the desired therapeutic effect. The primary cells can be obtained directly from a donor, a culture of cells from a donor, or from established cell culture lines. Cells may be obtained from the same or a different species than the subject to be treated, but preferably are of the same species, and more preferably of the same immunological profile as the subject. Such cells can be obtained, for example, by biopsy from a close relative or matched donor.

The progenitor cells are chosen for their ability to promote tissue production from the primary cells. Progenitor cells that can be used include, but are not limited to, progenitor cells from various types of tissues, such as mesenchymal stromal cells from bone marrow, endothelial progenitor cells, muscle progenitor cells (e.g., satellite cells), pancreatic progenitor cells, periosteum progenitor cells, neural progenitor cells, blast cells, intermediate progenitor cells, and stem cells, including stem cells from embryos, umbilical cord, or adult tissues, or induced pluripotent stem cells.

The use of progenitor cells to catalyze tissue production by the primary cells allows a smaller number of primary cells to be used for tissue production than would be needed if the primary cells were used alone in tissue production. In one embodiment, the number of tissue-specific primary cells included in compositions used for tissue production is the minimal number needed to promote a therapeutically effective amount of tissue production to treat a particular injury or disease involving tissue degeneration. In one embodiment, the percentage of primary cells in a mixed culture with progenitor cells is 1% or less.

In certain embodiments, one or more additional factors, such as nutrients, cytokines, growth factors, antibiotics, anti-oxidants, or immunosuppressive agents may be added to the scaffold to improve cell function or viability. The composition may also further comprise a pharmaceutically acceptable carrier.

Exemplary growth factors include, fibroblast growth factor (FGF), insulin-like growth factor (IGF), transforming growth factor beta (TGF-β), epiregulin, epidermal growth factor (“EGF”), endothelial cell growth factor (“ECGF”), nerve growth factor (“NGF”), leukemia inhibitory factor (“LIF”), bone morphogenetic protein-4 (“BMP-4”), hepatocyte growth factor (“HGF”), vascular endothelial growth factor-A (“VEGF-A”), and cholecystokinin octapeptide.

Exemplary immunosuppressive agents are well known and may be steroidal (e.g., prednisone) or non-steroidal (e.g., sirolimus (Rapamune, Wyeth-Ayerst Canada), tacrolimus (Prograf, Fujisawa Canada), and anti-IL2R daclizumab (Zenapax, Roche Canada). Other immunosuppressant agents include 15-deoxyspergualin, cyclosporin, methotrexate, rapamycin, Rapamune (sirolimus/rapamycin), FK506, or Lisofylline (LSF).

One or more pharmaceutically acceptable excipients may also be included. Exemplary excipients include, without limitation, carbohydrates, inorganic salts, antimicrobial agents, antioxidants, surfactants, buffers, acids, bases, and combinations thereof.

For example, an antimicrobial agent for preventing or deterring microbial growth may be included. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof. Antibmicrobial agents also include antibiotics that can also be used to prevent bacterial infection. Exemplary antibiotics include amoxicillin, penicillin, sulfa drugs, cephalosporins, erythromycin, streptomycin, gentamicin, tetracycline, chlarithromycin, ciproflozacin, azithromycin, and the like. Also included are antifungal agents such as myconazole and terconazole.

Various antioxidants can also be included, such as molecules having thiol groups such as reduced glutathione (GSH) or its precursors, glutathione or glutathione analogs, glutathione monoester, and N-acetylcysteine. Other suitable anti-oxidants include superoxide dismutase, catalase, vitamin E, Trolox, lipoic acid, lazaroids, butylated hvdroxyanisole (BHA), vitamin K, and the like.

Excipients suitable for injectable compositions include water, alcohols, polyols, glycerin, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

Acids or bases can also be present as an excipient. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C., 2000.

In certain embodiments, the invention includes compositions for generating new cartilage comprising adipose-derived stem cells and neonatal articular chondrocytes encapsulated in a hydrogel. In one embodiment, the hydrogel comprises chondroitin sulfate methacrylate (CS-MA) and poly(ethylene)glycol diacrylate (PEGDA). The neonatal articular chondrocytes, so encapsulated, produce cartilage in an amount effective for treatment of a subject in need of repair or replacement of cartilage, such as caused by a traumatic injury or a disease involving cartilage degeneration.

The adipose-derived stem cells and neonatal articular chondrocytes can be combined as a mixed culture or a bilayered culture in the hydrogel. In certain embodiments, adipose-derived stem cells and neonatal articular chondrocytes are combined in a mixed culture, wherein the ratio of adipose-derived stem cells to neonatal articular chondrocytes is about 25:75, about 50:50, about 75:25, about 90:10, about 95:5, about 99:1, or any ratio in between. In another embodiment, the percentage of neonatal articular chondrocytes in the mixed culture is 1% or less. In one embodiment, the number of neonatal articular chondrocytes is the minimal number needed to promote a therapeutically effective amount of cartilage production to treat an injury or disease involving cartilage degeneration.

The compositions, described herein, for transplanting cells are typically, though not necessarily, administered by injection or surgical implantation into the region requiring tissue replacement or repair. For example, compositions capable of producing new cartilage in a subject can be administered locally into an area of cartilage damage or loss, such as a damaged joint or other suitable treatment site of the subject.

The compositions of the invention, comprising progenitor cells and primary cells, can be used for treating a subject for tissue damage or loss, such as caused by a traumatic injury or a disease involving tissue degeneration. For example, the compositions comprising adipose-derived stem cells and neonatal articular chondrocytes can be used for treating a subject for cartilage damage or loss, such as caused by a traumatic injury or a disease involving cartilage degeneration.

In one embodiment, the invention includes a method for treating a subject for tissue damage or loss comprising administering a therapeutically effective amount of a composition comprising progenitor cells and tissue-specific primary cells, encapsulated in a three-dimensional scaffold, to the subject. By “therapeutically effective dose or amount” of a composition comprising progenitor cells and tissue-specific primary cells is intended an amount that, when administered as described herein, brings about a positive therapeutic response in a subject having tissue damage or loss, such as an amount that results in the generation of new tissue at a treatment site.

For example, a therapeutically effective dose or amount of a composition comprising adipose-derived stem cells and neonatal articular chondrocytes is intended an amount that, when administered as described herein, brings about a positive therapeutic response in a subject having cartilage damage or loss, such as an amount that results in the generation of new cartilage at a treatment site (e.g., a damaged joint). For example, a therapeutically effective dose or amount could be used to treat cartilage damage or loss resulting from a traumatic injury or a degenerative disease, such as arthritis or other disease involving cartilage degeneration. Preferably, a therapeutically effective amount restores function and/or relieves pain and inflammation associated with cartilage damage or loss.

Any of the compositions described herein may be included in a kit. The kit may comprise one or more containers holding the implant comprising the three-dimensional scaffold containing the encapsulated primary cells and progenitor cells or primary cells and conditioned media from progenitor cells. Alternatively, the kit may comprise the individual components needed for preparing an implant, such as the reagents for generating the three-dimensional scaffold, progenitor cells, primary cells, media, and optionally one or more other factors, such as growth factors, ECM components, antibiotics, and the like). Suitable containers for the compositions include, for example, bottles, vials, syringes, and test tubes. Containers can be formed from a variety of materials, including glass or plastic. A container may have a sterile access port (for example, the container may be a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

The kit can also comprise a package insert containing written instructions for methods of treating tissue damage or loss, such as caused by a traumatic injury or a disease involving tissue degeneration. The package insert can be an unapproved draft package insert or can be a package insert approved by the Food and Drug Administration (FDA) or other regulatory body.

In certain embodiments, the kit comprises a hydrogel composition for generating new cartilage, as described herein, or reagents and cells for preparing such a composition (e.g., chondroitin sulfate methacrylate (CS-MA), poly(ethylene)glycol diacrylate (PEGDA), adipose-derived stem cells, neonatal articular chondrocytes, media, and optionally one or more other factors, such as growth factors, ECM components, antibiotics, and the like). The kit may also comprise means for delivering the composition to a subject and instructions for treating a traumatic injury or a disease involving cartilage degeneration.

III. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLE 1

### Adipose-Derived Stem Cells Catalyze Cartilage Formation by Neonatal Articular Chondrocytes

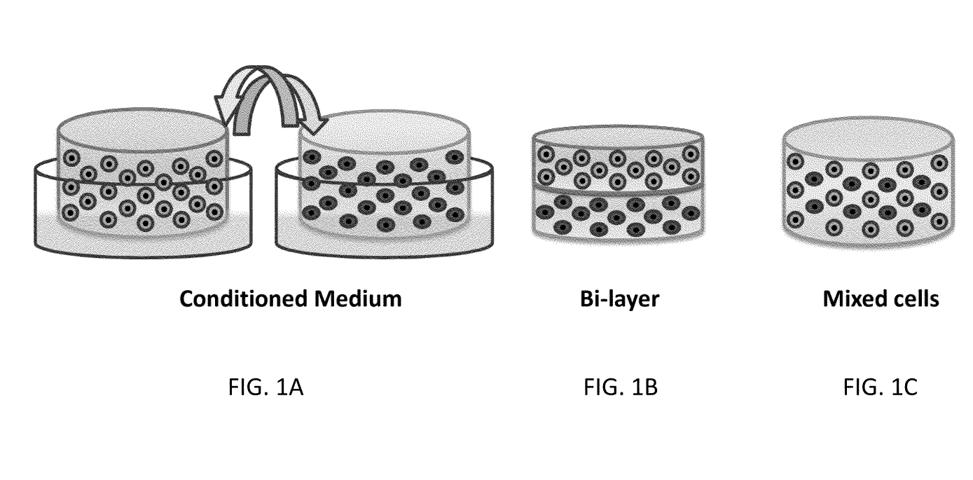

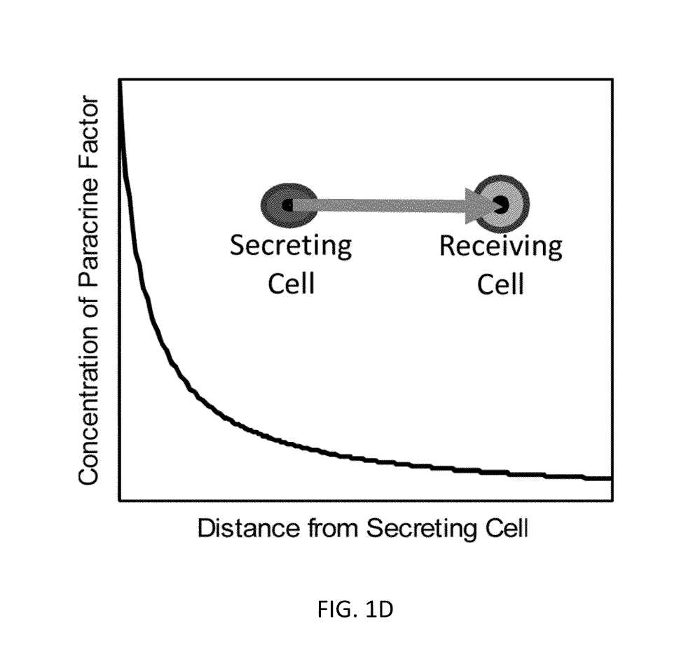

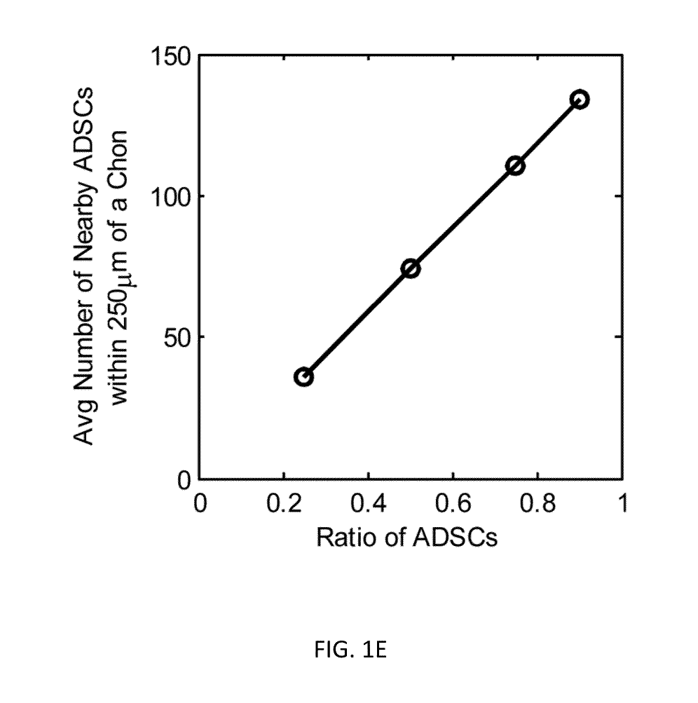

Here we report the use of adipose-derived stem cells (ADSCS) to catalyze cartilage formation by neonatal articular chondrocytes (CHONS) for cartilage regeneration. In order to examine the interaction between ADSCS and CHONS, three different in vitro culture models were used: 1) cells cultured with supplementation of conditioned medium from the other cell type (FIG. 1A), 2) bi-layered culture that confines the two cell types in separate layers mixed co-culture at different cell ratios (FIG. 1B), and 3) mixed culture of the two cells at different cell ratios (FIG. 1C). These culture models are designed specifically to allow for cells to interact at different levels of proximity, which is a governing parameter in cell-cell communication. The concentration of paracrine factors secreted by a cell decays exponentially with distance from the secreting cell (FIG. 1D), and the effective communication distance over which a cell can propagate soluble signal is within 250 μm (Francis and Palsson (1997) Proc. Natl. Acad. Sci. U.S.A. 94:12258-1262). In the mixed cell culture model, increasing ADSC ratio while keeping the overall cell density constant leads to a linear increase in the number of ADSCS that are within the effective communication distance of a CHON

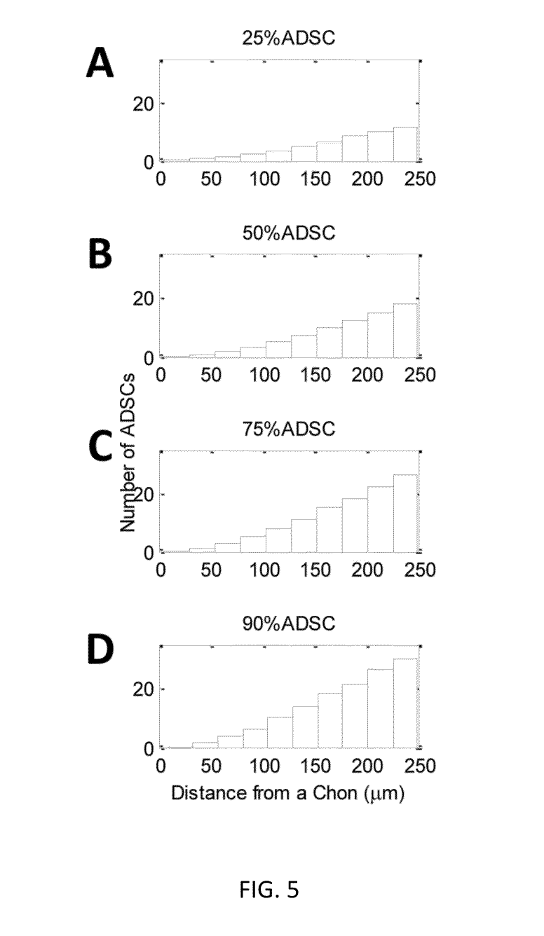

(FIG. 1E and FIG. 5).

In all the culture models, cells were encapsulated in a 3D biomimetic hydrogel consisting of chondroitin sulfate methacrylate (CS-MA) and poly(ethylene)glycol diacrylate (PEGDA), which enables enzymatic degradation and matrix turnover by cell-secreted chondroitinase (Varghese et al. (2008) Matrix Biol. 27:12-21; herein incorporated by reference). Human adult ADSCS and bovine neonatal CHONS were encapsulated in 3D biomimetic hydrogels and cultured in vitro for 21 days in chondrogenic medium supplemented with TGF-β3.

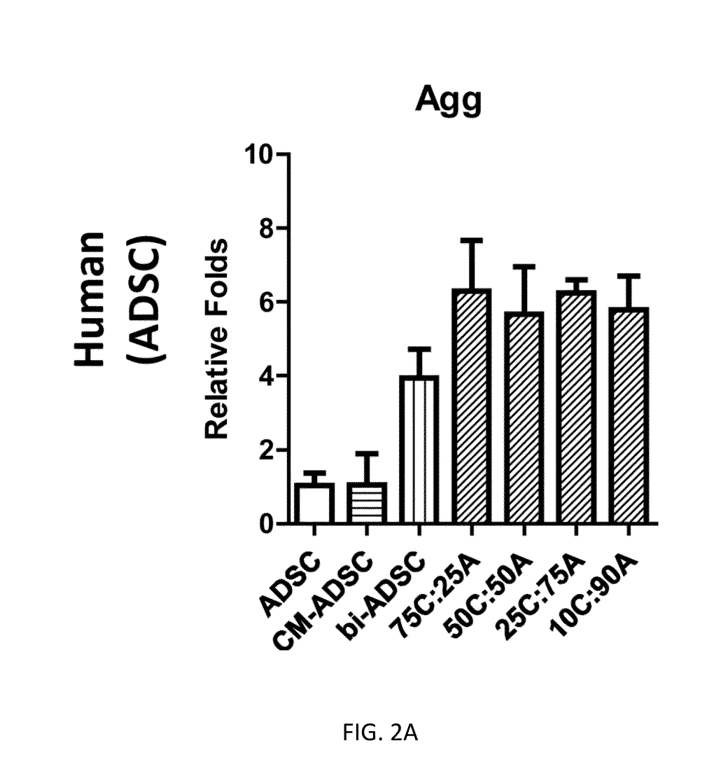

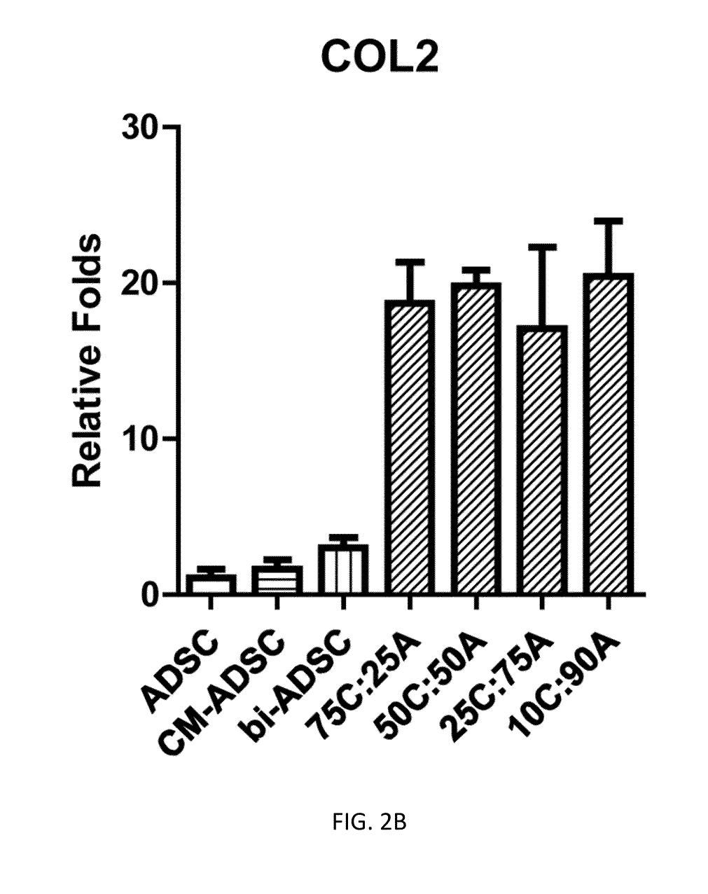

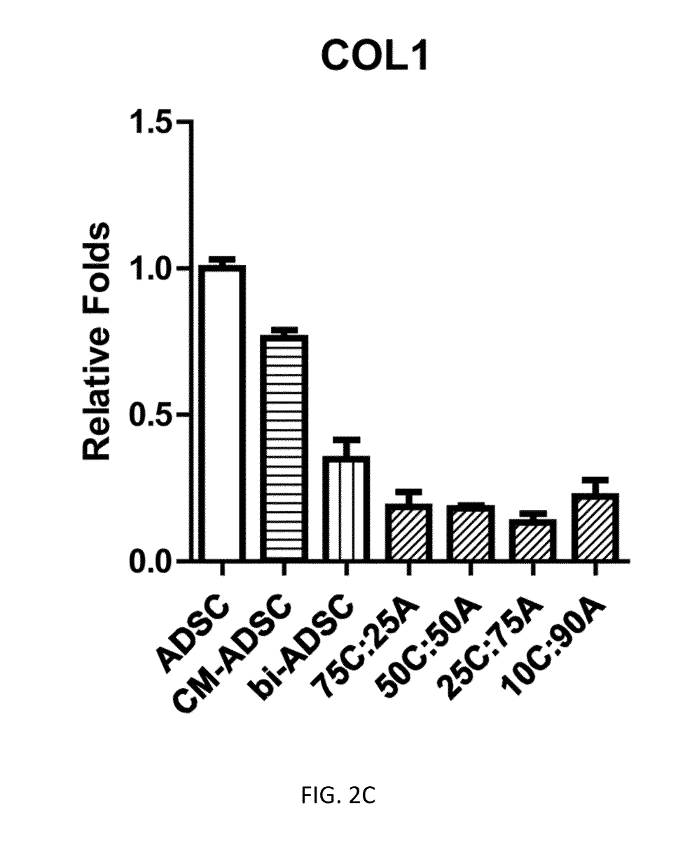

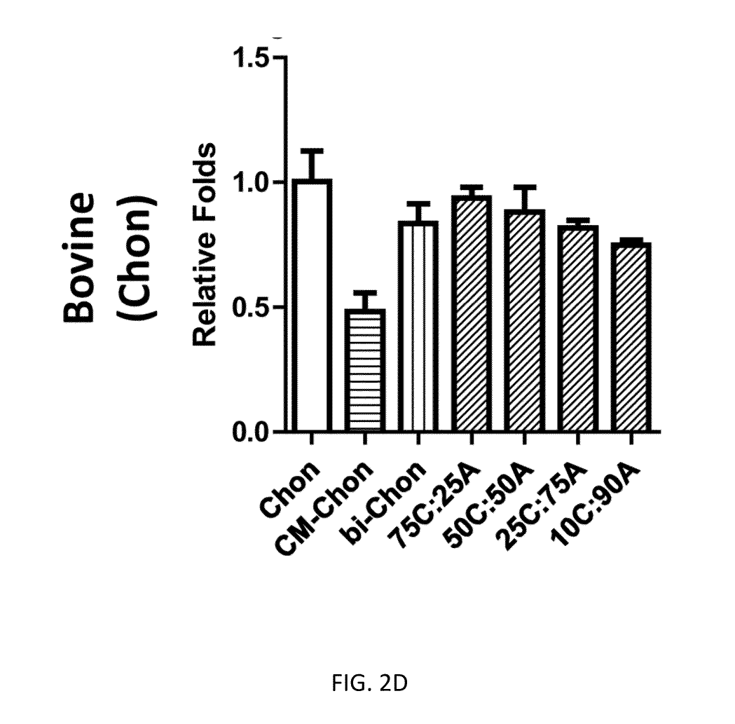

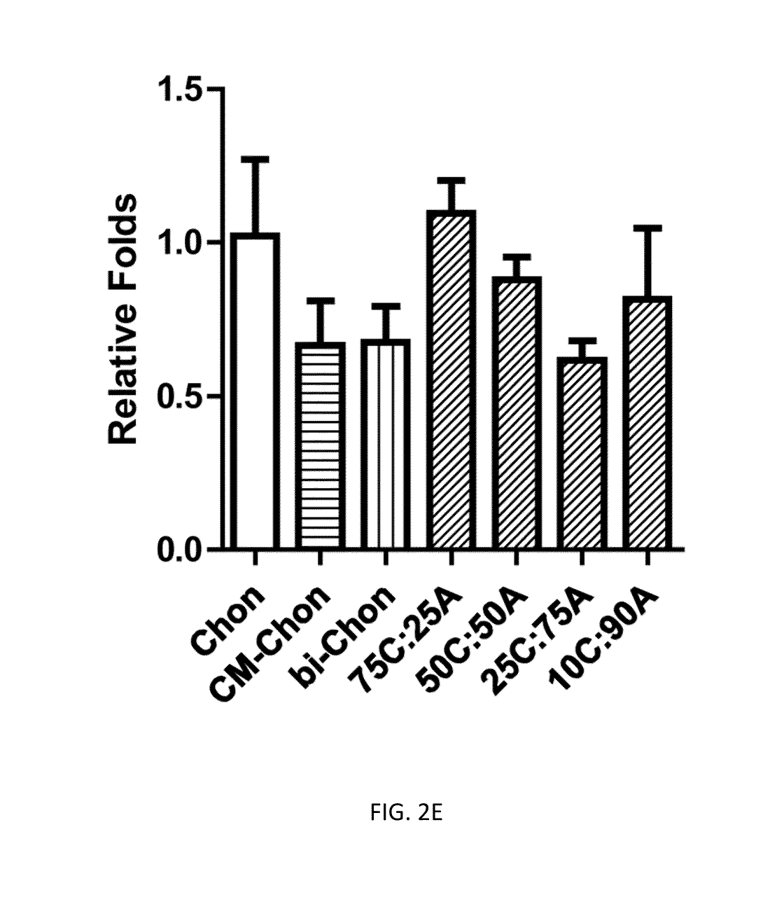

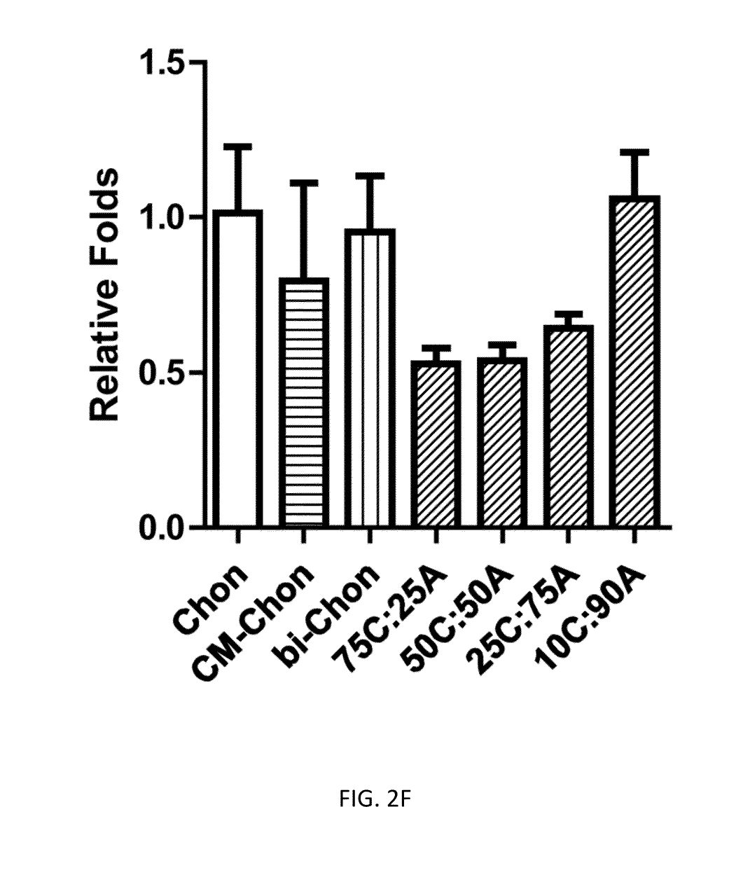

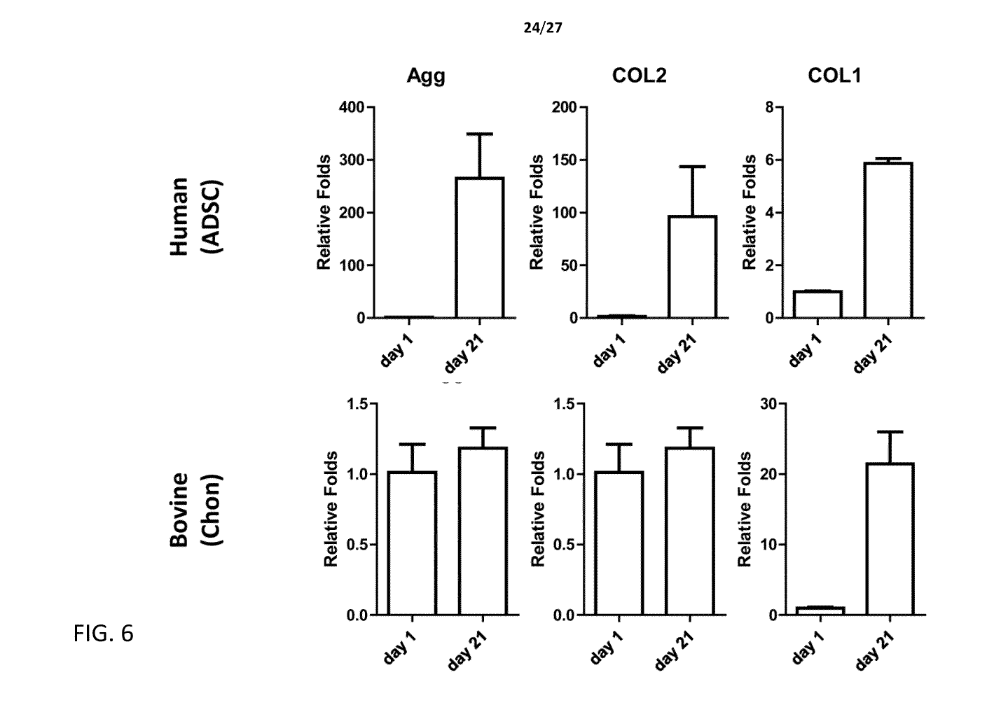

Gene markers associated with chondrocytes were evaluated with reverse transcriptase polymerase chain reaction (RT-PCR). To distinguish the fate of each cell type, specie-specific primers were used. At day 21, gene expression of aggrecan (Agg) and type II collagen (COL2) of ADSCS increased 265- and 96-fold respectively (FIGS. 6A and 6B), indicating that ADSCS underwent chondrogenesis under the induction of TGF-β3. ADSCS treated with conditioned medium collected from CHONS (CM-ADSC) resulted in an increase in chondrogenic gene expression (Agg and COL2) and a decrease in the fibroblastic marker, type I collagen (COL1) compared to the ADSC control (FIG. 2C). When ADSCS were cultured with CHONS in a bi-layered hydrogel (bi-ADSC), which allowed for a higher concentration as well as a dynamic exchange of paracrine factor compared to CM-ADSC, chondrogenic expression (Agg and COL2) was further increased while fibroblastic expression (COL1) was further decreased. As for CHONS, both treatment with conditioned medium collected from ADSCS (CM-CHON) and the bi-layer culture (bi-CHON) did not lead to significant changes in Agg, COL2, and COL1 expression (FIGS. 2D, 2E, and 2F).

Mixed cell culture, which allows for the two types to interact at close proximity, led to a marked increase in chondrogenic expression in ADSCS, maintenance of chondrocyte phenotype in CHONS, and reduction of fibroblastic expression in both cell types. Interestingly, changing cell ratios in the mixed co-culture did not lead to significant changes in chondrogenic gene expression. Agg and COL2 expression in the mixed co-culture groups exhibited 1400-1600 and 1500-1800 fold increases, respectively, over 21 days, which were 5.5-6 and 15.9-19 times higher than those of the ADSC control at day 21 (FIGS. 2A and 2B). COL1 expression was reduced compared to the ADSC control (FIG. 2C). As for CHONS, chondrogenic gene expression in mixed co-culture groups was maintained at levels similar to the CHON control (FIGS. 2D and 2E). Agg expression of CHONS in all the mixed co-culture groups except for 10C:90A was comparable to that of the CHON control. Similarly, COL2 expression of CHONS in the mixed co-culture groups was comparable to that of the CHON control. COL2 expression of the 25C:75A group was significantly lower than that of 75C:25A. COL1 expression in all the mixed co-culture groups except 10C:90A was reduced to approximately 50% of that of the CHON control (FIG. 2F).

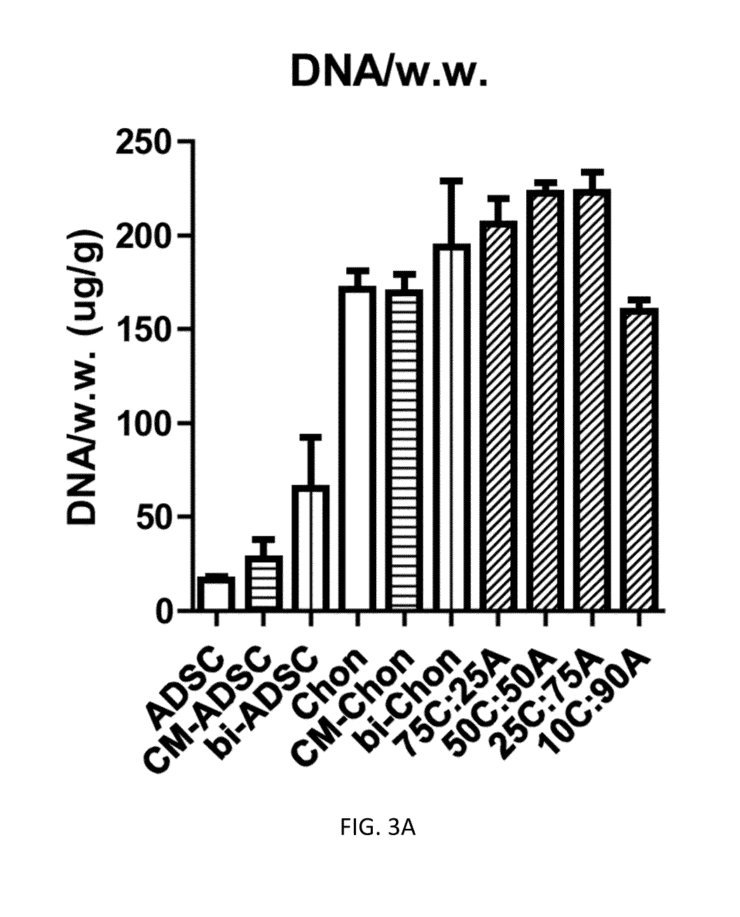

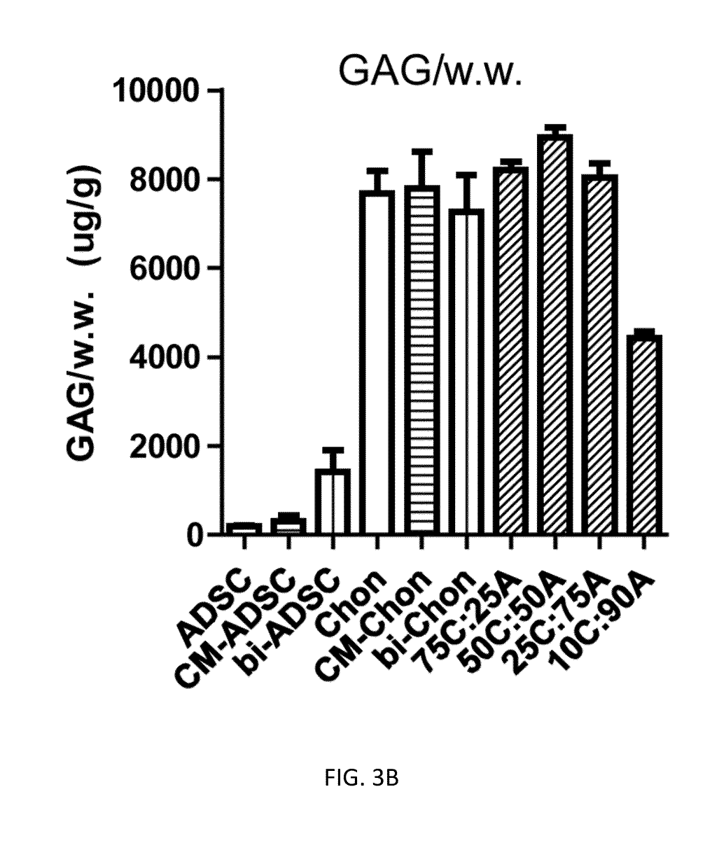

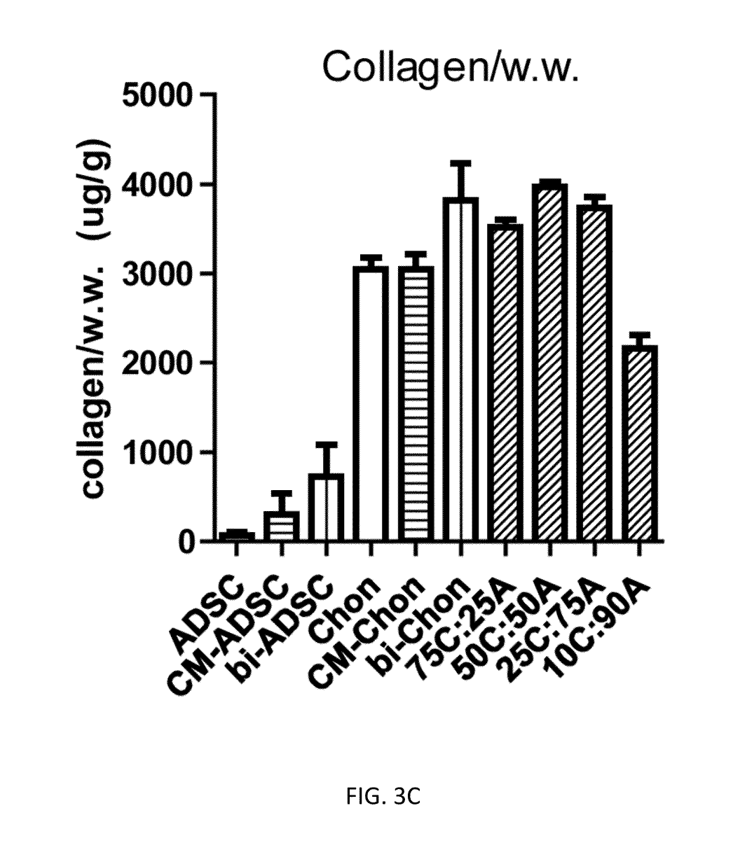

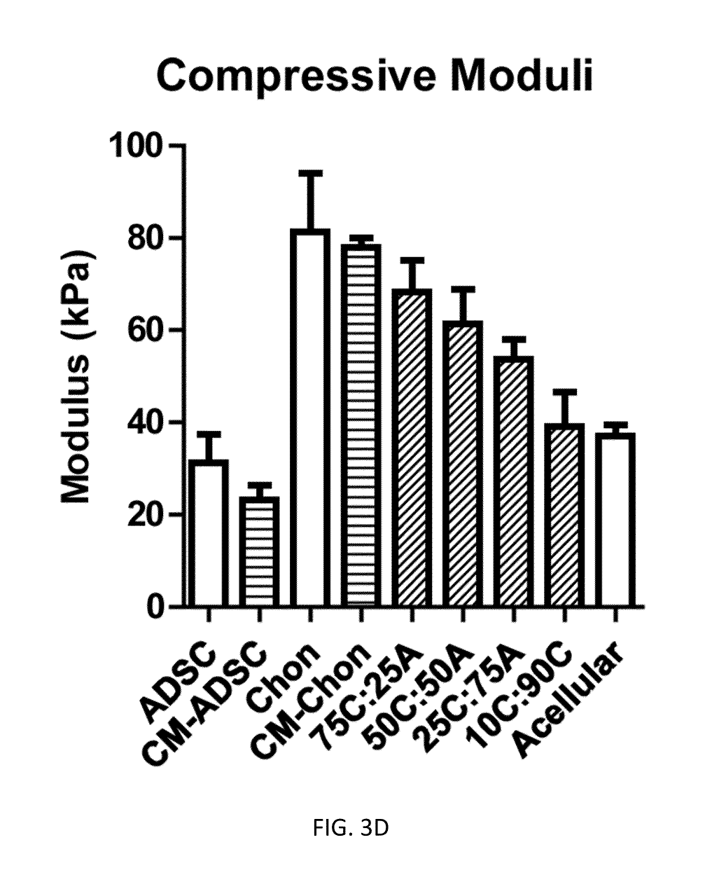

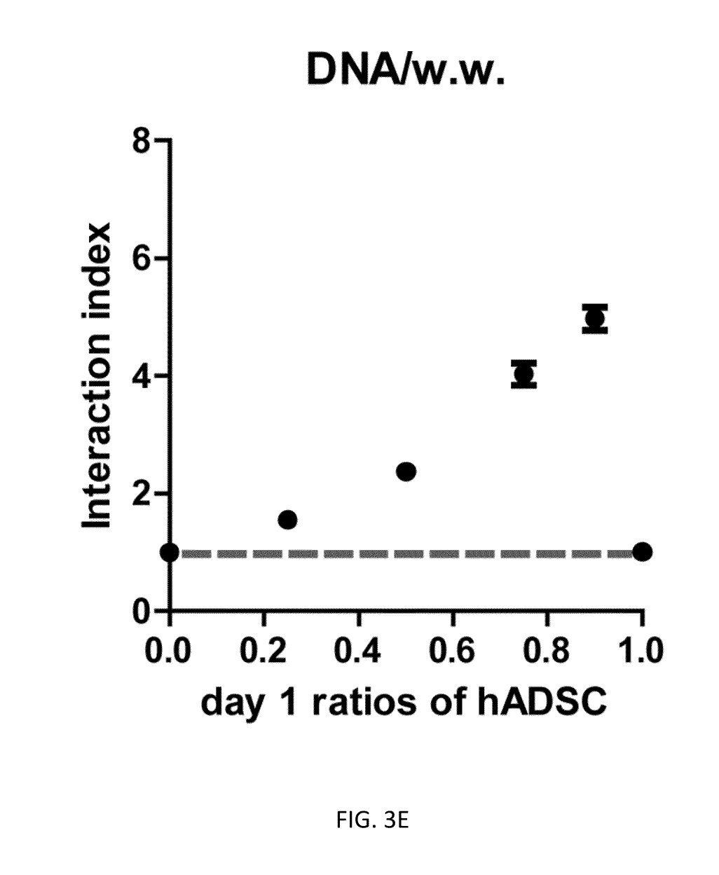

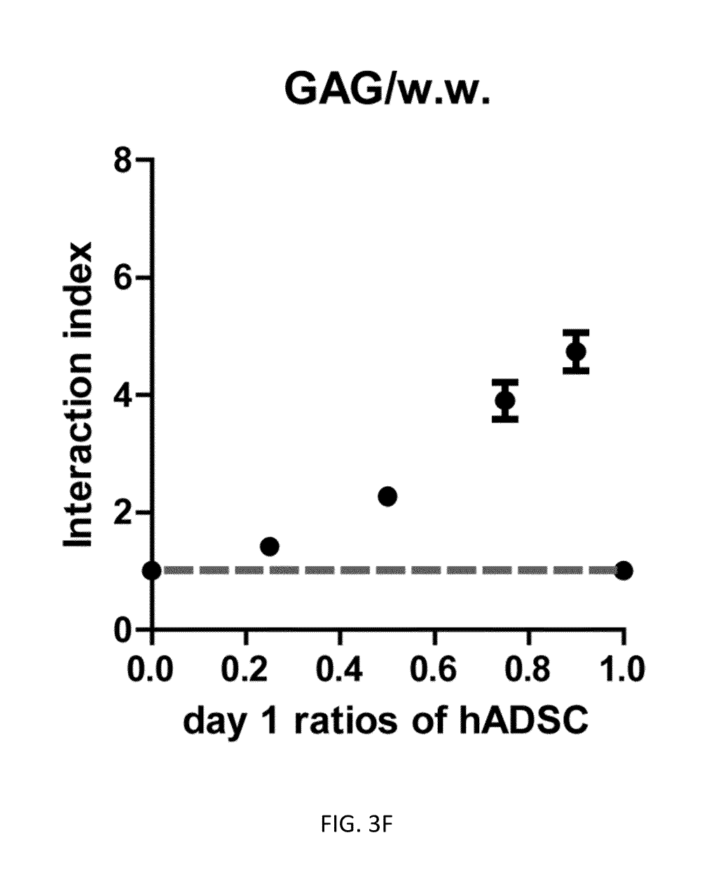

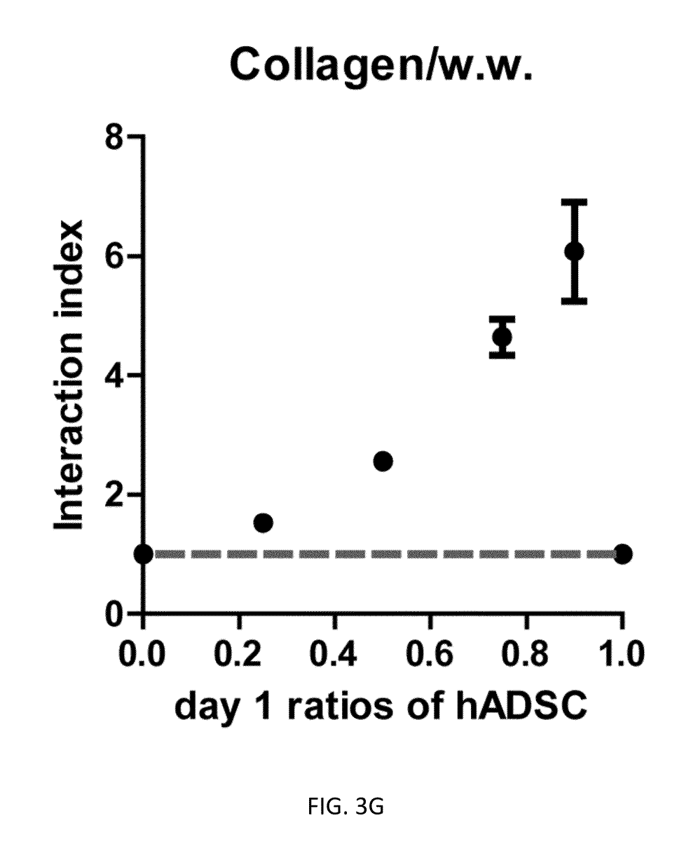

Next, we quantified cell proliferation and cartilage matrix production. To quantify cell proliferation over time, DNA contents were measured at day 1 and 21. In the ADSC control group, DNA content at day 21 was reduced to 29% of day 1 DNA content (FIG. 3A). Both conditioned medium and bi-layer co-culture had significantly higher numbers of ADSCS than that of the ADSC control at day 21. To quantify cartilage matrix production, sulfated glycosaminioglycan (sGAG) and total collagen content were measured at day 21. SGAG and collagen per wet weight exhibited similar trends (FIGS. 3B and 3C). ADSC cultured in conditioned medium led to approximately 1.6- and 3-fold increases in sGAG and collagen per wet weight respectively compared to the ADSC control group. The effect of bi-layer culture on ADSC matrix production was more significant, resulting in 7.6 and 10.4 fold increases in sGAG and collagen per wet weight respectively. As for CHONS, cell number and sGAG content per wet weight were maintained in conditioned medium and bi-layer culture (FIGS. 3A and 3B). Collagen content per wet weight in the conditioned medium group was similar to that of the CHON control group, but was significantly increased in the bi-layer group (bi-CHON) (FIG. 3C).

Mixed cell culture led to significantly higher cell number and cartilage matrix content than ADSC control. Gross appearance of the freeze-dried cell-hydrogel constructs indicated that matrix was formed in all the mixed co-culture groups and the CHON control group after 21 days of culture (FIG. 6), while the freeze-dried construct of the ADSC control group remained similar in size overtime. In all the mixed cell groups and CHON control group, DNA per wet weight increased significantly (about 3.1 to 4.2-fold) over 21 days of culture (FIG. 3A). The variations in sGAG and collagen content per wet weight with changes in cell ratios exhibited similar trends (FIGS. 3B and 3C). Of all the cell ratios examined, the 50C:50A group resulted in the most cartilage matrix formation, reaching up to 30% higher sGAG and collagen content per wet weight than the CHON control group. Surprisingly, mixed cell culture with as low as 25% CHONS (25C:75A) resulted in higher sGAG (-18%) and collagen (about 22%) per wet weight than CHONS alone. When the percentage of CHONS in mixed co-culture was further reduced (10C:90A), sGAG and collagen content per wet weight dropped significantly to approximately 59 and 71% of CHON control group respectively. Elastic modulus in CHON control and all the mixed co-culture groups increased significantly over 21 days (FIG. 3D). At day 21, the modulus was the highest in the CHON control group and decreased progressively with an increase in ADSC ratio in the co-culture population.

To quantify the effects of cell ratio variation on cell proliferation and cartilage matrix production, the measured DNA, sGAG, and collagen content were compared against the expected values. At each cell ratio, the interaction index, which is the measured matrix content (DNA, sGAG, or collagen) in the mixed co-culture group normalized by the expected matrix content based on the measured matrix content in the CHON and hADSC alone groups, was calculated as previously shown by Acharya et al. (Acharya et al. (2012) J. Cell Physiol. 227:88-97; herein incorporated by reference). The interaction for DNA, sGAG, and collagen per wet weight in all the mixed co-culture groups were higher than 1 (FIGS. 3E, 3F, and 3G). Interestingly, the interaction index increased with an increase ratio of ADSCS in the mixed co-culture population, indicating that the extent of cell proliferation and cartilage matrix production was highly dependent on the ratio of the two cell types. At 90% ADSC (10C:90A), DNA, sGAG, and collagen content per wet weight were approximately 5-6-fold higher than expected. When normalized by DNA, however, the interaction index for collagen and sGAG was close to 1 (not shown), indicating that sGAG and collagen production were not increased significantly on a per cell basis. This suggests that mixed cell culture enhanced cartilage production primarily through the stimulation of cell proliferation. Remarkably, although an inverse relationship between cell proliferation and matrix production per cell has been reported in the literature (Detamore and Athanasiou (2004) Arch. Oral Biol. 49:577-583), in our study the increased cell proliferation as a result of mixed cell culture did not reduce matrix production on a per cell basis.

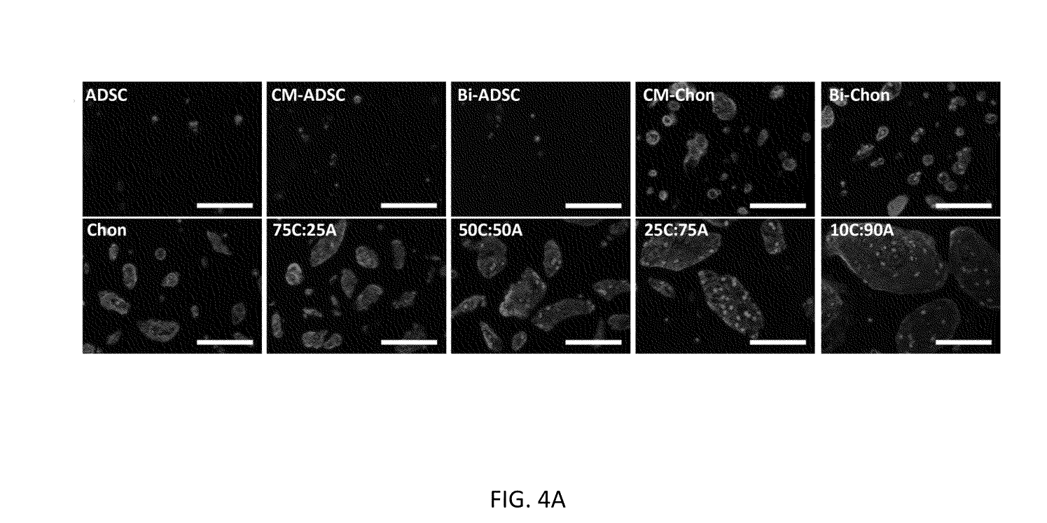

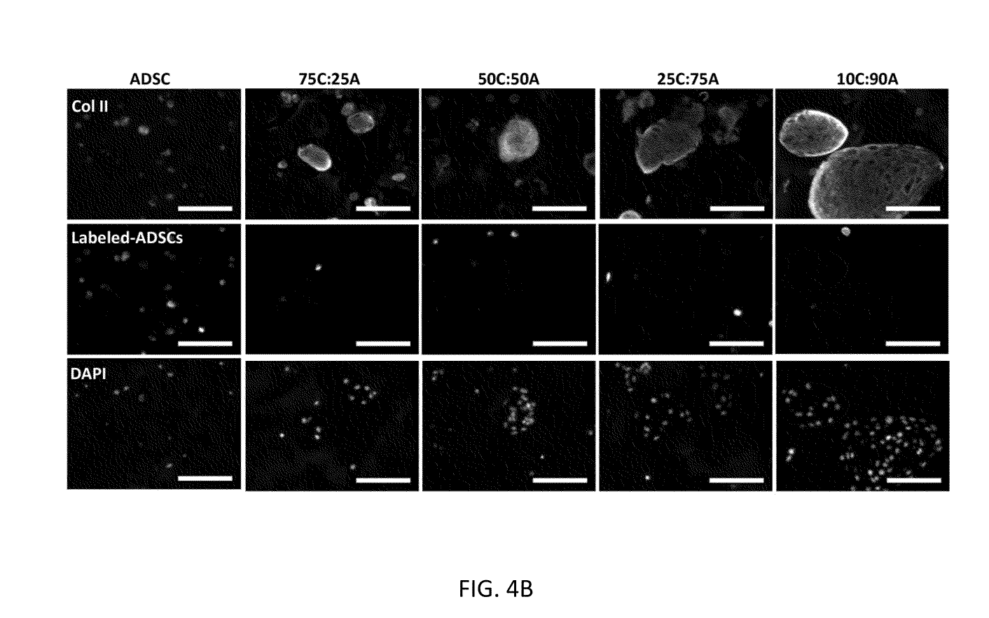

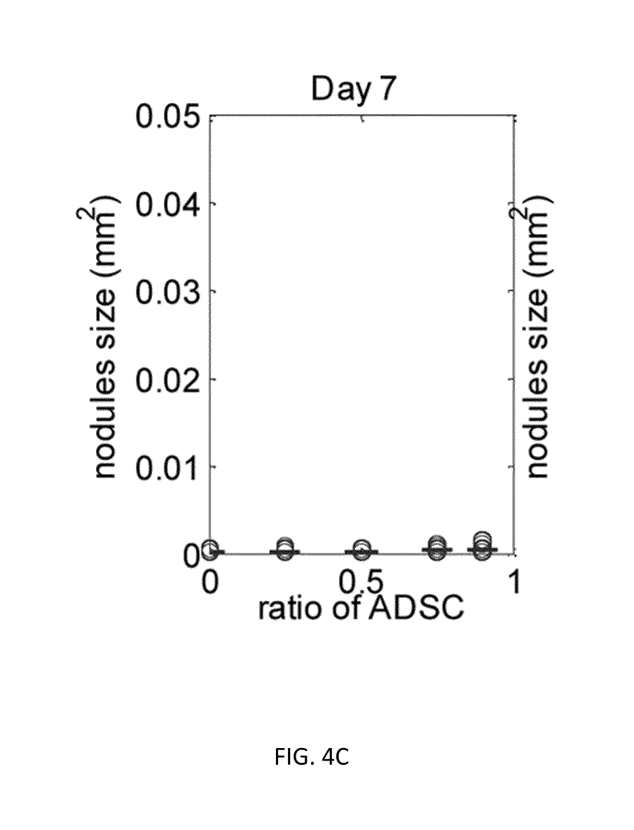

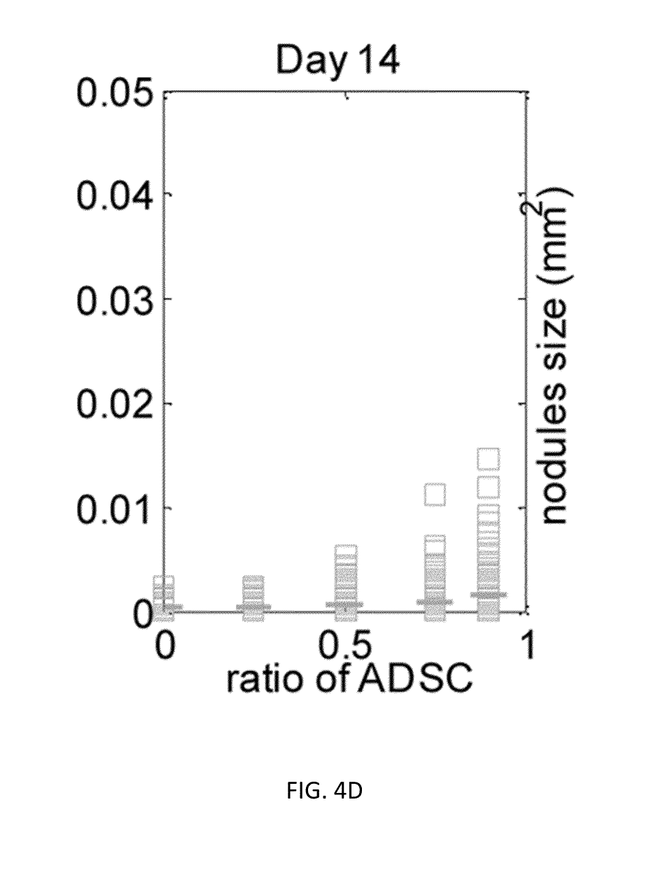

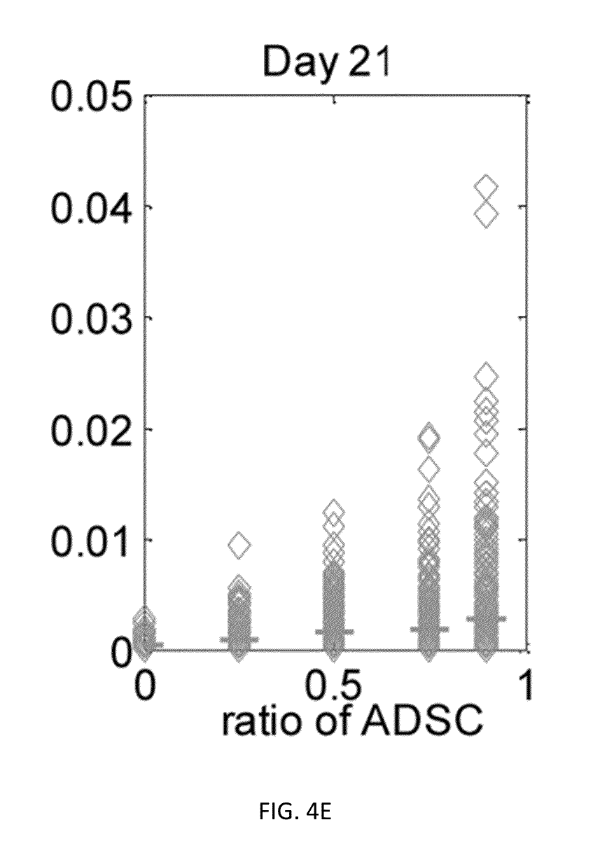

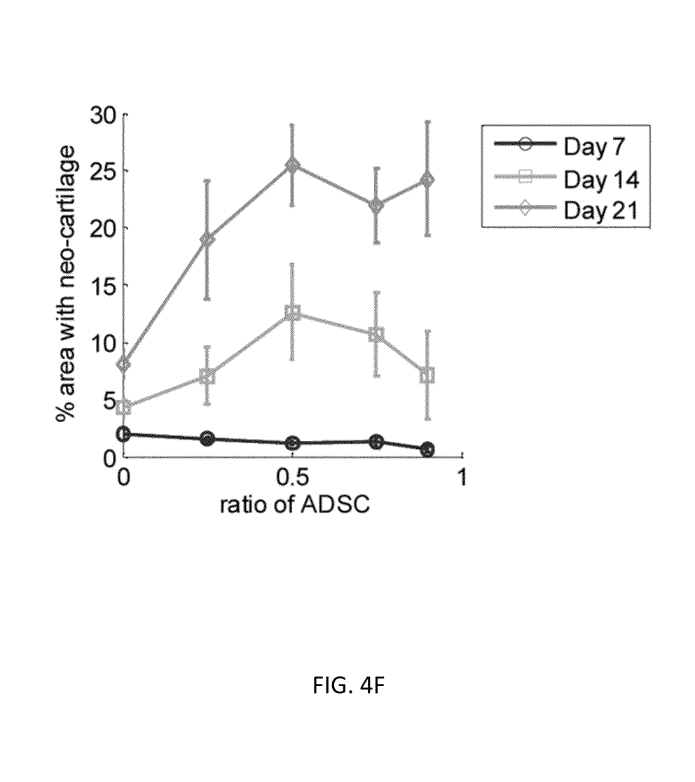

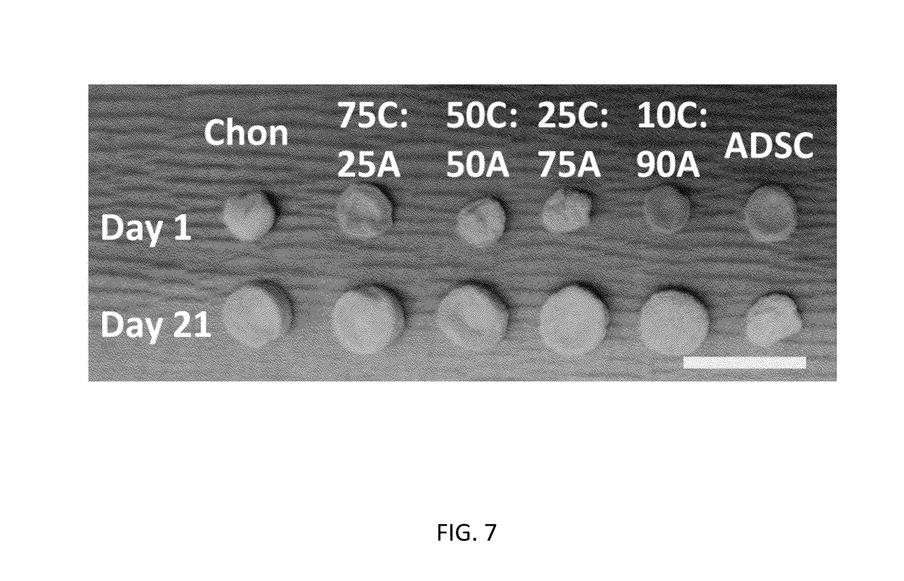

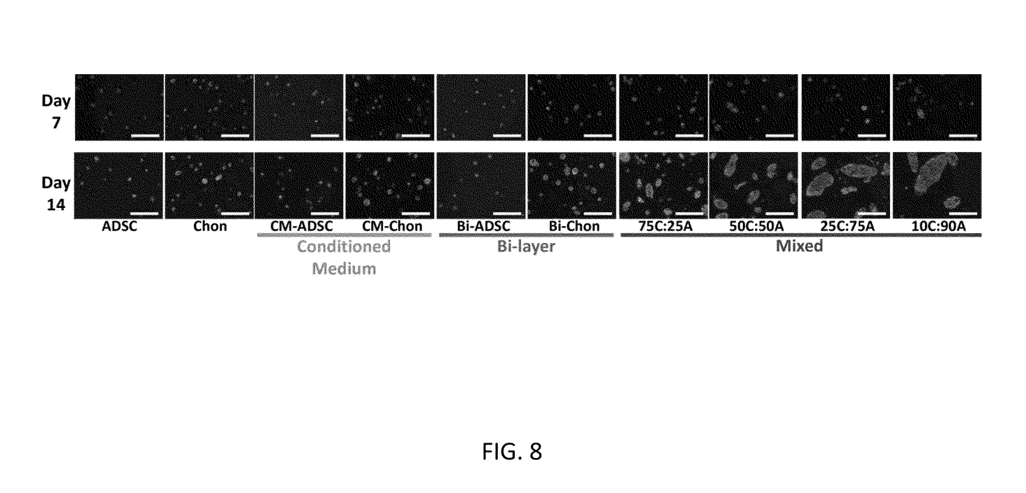

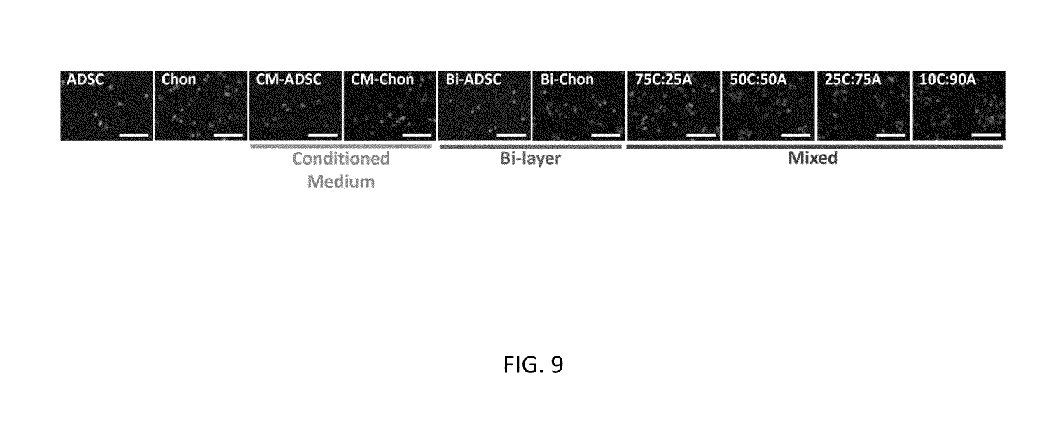

In addition to the extent of cell proliferation and cartilage matrix production, the differential effects of conditioned medium, bi-layer, and mixed cell culture on cartilage matrix production were also evident in the spatial organization of neo-cartilage within the 3D hydrogels as shown by type II collagen immunostaining Conditioned medium and bi-layer culture did not lead to obvious changes in type II collagen production for both cell types. On the contrary, variation in cell ratios in the mixed cell culture led to differential formation and spatial organization of neo-cartilage nodules within the 3D hydrogels. While cells appeared to distribute evenly in the hydrogel matrix at day 7 (FIG. 7), cell aggregates and neo-cartilage nodules were observed at day 14 (FIG. 7) and 21 (FIG. 4A). Interestingly, the individual nodule size as well as the total area occupied by the nodules increased with an increase in ADSC ratio (FIGS. 4C-4E); at day 21, the nodule size in the group with 90% ADSCS (10C:90A) was 6 times larger than that in bAC alone group. It is also worth noting that while the mixed co-culture group with 90% hADSCS (10C:90A) was remodeled extensively with large neo-cartilage nodules, the control group with 100% ADSCS exhibited little cartilage deposition (FIG. 4A). Type I collagen staining was minimal (FIG. 9) in all groups, indicating that the cartilage nodules produced were hyaline cartilage instead of fibrocartilage. The drastic differences in neo-cartilage organization in the mixed cell culture demonstrated the cell fate as well as tissue formation was tightly regulated by the dynamic cell-cell interactions between CHONS and ADSCS. Using a 3D biomimetic hydrogel culture system enable us to observe the spatial and temporal differences in hydrogel remodeling and cartilage matrix organization at different cell ratios.

To further examine the distribution and the relative contribution of each cell type to cartilage nodule formation in the mixed co-culture, ADSCS were fluorescently labeled with lipophilic membrane dyes prior to encapsulation in hydrogels. Fluorescently labeled ADSCS were distributed throughout the hydrogel matrix at different time points, while aggregates of CHONS (negatively labeled cells) were observed on day 14 and 21. Direct cell-cell contact between the two cell types was not evident. Cell tracking along with co-localization of collagen II immunostaining in mixed cell culture indicated that cartilage nodules were formed primarily by aggregates of CHONS (FIG. 4B). This is in agreement with recent studies that showed that BMSCs stimulated CHON proliferation and cartilage matrix production (Acharya et al., supra; Wu et al. (2012) Tissue Eng. Part A. 18:1542-1551; Meretoja et al. (2012) Biomaterials 33:6362-6369). The lack of ADSCS within the cartilage nodules in mixed co-culture hydrogels, together with the increase in the size of these nodules with an increase in ADSC ratio in the mixed co-culture, suggested that the two cell types interact through paracrine signaling in a dose-dependent manner. With an increase in ADSCS in the mixed co-culture system, the number of ADSCS within effective communication distance of the CHONS increased, which stimulated CHONS to proliferate and produce larger cartilage nodules. It has been shown that ADSCS secrete growth factors such as fibroblast growth factor-2 (FGF-2), vascular endothelial growth factor (VEGF), hepatocyte growth factor (HGF), insulin-like growth factor 1(IGF-1), which are known to stimulate cell proliferation. Of these factors, FGF-2 and IGF-1 have been shown to induce GAG and type II collagen synthesis in chondrocytes (Veilleux et al. (2005) Osteoarthritis Cartilage 13:278-286). In addition, FGF-2 has also been shown to reduce fibroblastic and hypertrophic phenotype in chondrocytes (Kato et al. (1990) J. Biol. Chem. 265:5903-5909; Martin et al. (2001) J. Cell Biochem. 83:121-128). It has been shown that the differentiation state of stem cells may impact their role as a stimulator for tissue formation. For instance, Rothenberg et al. showed that BMSCs that were pre-differentiated towards osteogenic lineage for 3 days acted as a stronger stimulator for cartilage tissue formation when co-cultured with chondrocytes than naïve BMSCs (Rothenberg et al. (2011) Stem Cells Dev. 20:405-414). In our culture system, ADSCS differentiated towards chondrogenic lineage under the induction of TGF-β3, as indicated by increase in chondrogenic gene expression (Agg and COL2). The differentiation state of ADSCS may directly affect paracrine factors secretion by ADSCS, which in turn influence the interaction between ADSCS and CHONS.

The dependence of cell proliferation and matrix synthesis on cell ratios in mixed cell culture along with the relatively weak response in conditioned-medium and bi-layered co-culture strongly suggests that local concentration and distribution of paracrine factors play a crucial role in mediating cell-cell crosstalk and the subsequent neo-tissue formation. In native tissue, the extracellular matrix (ECM) mediates soluble signaling through the storage, binding, presentation, and presentation of soluble growth factors. Growth factors secreted by cells may diffuse through the tissue, be internalized by neighboring cells, get physically entrapped within the matrix, or bind to specific ECM proteins. Growth factors that are known to mediate in chondrocyte metabolism such as FGFs, IGFs, and TGF-β's have been shown to bind to the ECM (van der Kraan et al. (2002) Osteoarthritis Cartilage 10:631-637; Taipale and Keski-Oja (1997) FASEB J. 11:51-59). Binding these cell-secreted growth factors to the ECM modulates the dynamics of autocrine and paracrine signaling, creating high local concentrations and limiting the diffusion of these factors within the ECM. Similarly, in the 3D hydrogel culture in this study, interactions of the paracrine factors with the hydrogel matrix as well as the newly synthesized cartilage ECM may result in localization of these factors in the hydrogel construct, limiting the amount of soluble factors that diffused into the media. This explains the differential results observed in mixed vs. bi-layer or conditioned medium culture. Likewise, the relatively weak response observed in our conditioned medium culture as compared to transwell co-culture reported in other studies may be due to the fact that a large portion of the paracrine signals were retained in the cell-gel construct as opposed to diffusing out into the media (Aung et al. (2011) Arthritis Rheum. 63:148-158).

Overall, this study clearly demonstrated that ADSCS catalyzed cell proliferation and cartilage formation by neonatal CHONS in a dose-dependent manner. At 21 days, mixed cell culture with as low as 25% ADSCS resulted in GAG and collagen content that were higher than those in CHON alone group. Although we only examined cartilage formation for up to 21 days, our immunostaining results indicated that neo-cartilage formation increased over the course of culture, and that the mixed co-culture groups with a high ratio of ADSC seem to catch up with the groups containing low ratio of ADSCS. Therefore, it is likely that at a later time point, neo-cartilage formation in the 10C:90A group would surpass the CHON alone as well as other mixed co-culture groups with lower ADSC ratios. It is expected that neo-cartilage deposited by the cells would completely replace the 3D hydrogel over a longer period of culture time, leading to the formation of a heterogeneous and mechanically functional neo-cartilage tissue.

The concept of utilizing stem cells to catalyze tissue formation by primary cells for tissue regeneration is not limited to application in cartilage but can be applied to other tissue types as well. By using different 3D biomimetic hydrogel culture models, we demonstrated the importance of intercellular distance and cell distribution in mediating the interactions of the two cell types, showing that the extent of cell proliferation and cartilage matrix production and organization were tightly regulated by these two variables. Our findings provide new insight into the design of 3D culture systems to probe cell-cell interactions, highlighting the advantages of using a 3D bio-mimetic hydrogel to examine cell-cell interactions in a physiologically relevant manner. In addition, our results also emphasized the relevance of manipulating cell-cell interactions in tissue engineering applications, highlighting the possibility of co-delivering small amount of neonatal chondrocytes from an autologous source with ADSCS to catalyze cartilage formation as a novel strategy to enhanced cartilage tissue repair and regeneration.

Materials and Methods

Cell Isolation and Culture

Chondrocytes: Hyaline articular cartilage was dissected from the femoropatellar groove of two stifle joints from a three-day old calf (Research 87, Marlborough, Mass.). The cartilage was sliced into thin pieces and digested in 1 mg/mL collagenase type II and type IV in high glucose DMEM supplemented with 100 U/mL penicillin and 0.1 mg/mL streptomycin for 24 hours at 37° C. The cell suspension was filtered through a 70 μm nylon mesh, washed in DPBS and centrifuged at 460 g for 15 minutes for three times, and counted with a hemocytometer. The bovine articular chondrocytes (bACs) were then suspended in freezing media (DMEM supplemented with 10% dimethyl sulfoxide (DMSO) and 50% fetal bovine serum (FBS), frozen at 1° C./minute, and stored in liquid nitrogen.

Adipose-derived stem cells: Adult human adipose-derived stem cells (ADSCS) were isolated from excised human adipose tissue with informed consent as previously described (Zuk et al. (2001) Tissue Eng. 7:211-228; herein incorporated by reference). ADSCS were expanded for 4 passages in high glucose DMEM supplemented with 5 ng/mL basic fibroblast growth factor (bFGF), 100 U/mL penicillin, and 0.1 mg/mL streptomycin.

3D Hydrogel Co-Culture

On the day of cell encapsulation, bACs were thawed, recounted and used without further expansion. Cells were suspended at 15×10⁶ cells/mL in a hydrogel solution consisted of 7% weight/volume (w/v) poly(ethylene glycol diacrylate) (PEGDA, MW=5000 g/mole), 3% w/v chondrointin sulfate-methacrylate (CS-MA), and 0.05% w/v photo-initiator (Irgacure D 2959; Ciba Specialty Chemicals) in DPBS. Cell-hydrogel suspension was pipetted into cylindrical gel mold with 75 μl volume and exposure to UV light (365 nm wavelength) at 3 MW/m² for 5 minutes to induce gelation. To create bi-layered hydrogel, cell-hydrogel suspension (37.5 μl each) of one cell type was deposited into the cylindrical gel mold and photo-crosslinked before deposition of the next cell-hydrogel layer. To prevent direct cell-cell contact, the two cell-hydrogel layers were separated by an acellular hydrogen layer (10 μl). The UV exposure times for the three sequential layers were 3, 2, and 5 minutes.

Co-Culture Models

To examine the effects of different local paracrine signals on cell fate, bACs and hADSCS were co-cultured in three different co-culuture models: (1) Mixed co-culture in a single-layered hydrogel in 4 mixing ratios of bACs and hADSCS (75C:25A, 50C:50A, 25C:75A, 10C:90A); bACs alone and hADSCS alone were included as controls; (2) bi-layered co-culture with equal number of bACs and hADSCS confined to its own layer (bi-bAC and bi-hADSC); and (3) each of the cell types encapsulated in 3D hydrogels alone with supplementation of conditioned medium from the other cell type encapsulated in 3D (CM-bAC and CM-hADSC). The conditioned medium was collected every two days, filtered through a 0.2 μm mesh, and diluted with an equal volume of fresh chondrogenic medium. All samples were cultured in chondrogenic medium (high-glucose DMEM containing 100 nM dexamethasone, 50 μg/ml ascorbate-2-phosphate, 40 μg/ml proline, 100 μg/ml sodium pyruvate, 100 U/mL penicillin, 0.1 mg/mL streptomycin, and ITS Premix (5 μg/ml insulin, 5 μg/ml transferrin, 5 ng/ml selenious acid, BD Biosciences)) supplemented with 10 ng/ml TGF-β3 for 3 weeks.

Gene Expression Analysis

Total RNA was extracted from cell-hydrogel constructs (n=3) using TRIzol and the RNeasy mini kit (Qiagen). One mg of RNA from each sample was reversed transcribed into cDNA using the Superscript First-Strand Synthesis System (Invitrogen). Real-time polymerase chain reaction (PCR) was performed on an Applied Biosystems 7900 Real-Time PCR system using SYBR green master mix (Applied Biosystems) with the primers listed in Table 1. Human- and bovine-specific primers were used to quantify gene expression of chondrogenic markers including Type II collagen (COL2) and aggrecan (Agg) as well as fibroblastic marker type I collagen (COL1) using ΔΔCt method. Gene expression levels were normalized internally to GAPDH. Relative fold changes represent changes in gene expressions compared with bACs alone group (for bovine-specific gene expressions) and hADSCS alone group (for human-specific gene expressions) at day 1.

TABLE 1
List of species-specific primers used for real-time polymerase chain
reaction.

| Gene | | | |
|---|---|---|---|
| Name | Species | Primer Sequence | GenBank No. |
| GAPDH | Human | F: 5′ CGCTCTCTGCTCCTCCTGTT 3′ (SEQ ID NO: 1) | NM_002046.3 |
| | | R: 5′ CCATGGTGTCTGAGCGATGT 3′ (SEQ ID NO: 2) | |
| | Bovine | F: 5′ AGATGGTGAAGGTCGGAGTG (SEQ ID NO: 3) | NM_001034034.1 |
| | | R: 5′ GATCTCGCTCCTGGAAGATG (SEQ ID NO: 4) | |
| Aggrecan | Human | F: 5′ TGAGGAGGGCTGGAACAAGTACC 3′ (SEQ ID NO: 5) | NM_001135.3 |
| (Agg) | | R: 5′ GGAGGTGGTAATTGCAGGGAACA 3′ (SEQ ID NO: 6) | |
| | Bovine | F: 5′ CACCACAGCAGGTGAACTAGA 3' (SEQ ID NO: 7) | NM_173981.2 |
| | | R: 5′ GCTTGCTCCTCCACTAATGTC 3′ (SEQ ID NO: 8) | |
| COL2A1 | Human | F: 5′ TCACGTACACTGCCCTGAAG 3′ (SEQ ID NO: 9) | NM_001844.4 |
| (COL2) | | R: 5′ TTGCAACGGATTGTGTTGTT 3′ (SEQ ID NO: 10) | |
| | Bovine | F: 5′ GTGGGGCAAGACTATGATCG 3′ (SEQ ID NO: 11) | NM_001113224.1 |
| | | R: 5′ TGCAATGGATTGTGTTGGTT 3′ (SEQ ID NO: 12) | |
| COL1A2 | Human | F: 5′ AGGGCAACAGCAGGTTCACTTACA 3′ (SEQ ID NO: 13) | NM_000089.3 |
| (COL1) | | R: 5′ AGCGGGGGAAGGAGTTAATGAAAC 3′ (SEQ ID NO: 14) | |
| | Bovine | F: 5′ ACATTGGCCCAGTCTGTTTC 3′ (SEQ ID NO: 15) | NM_174520.2 |
| | | R: 5′ GGGAGGGGGAGTGAATTAAA 3′ (SEQ ID NO: 16) | |

Biochemical Analysis

Cell-hydrogel constructs (n=4) were weighed wet, lyophilized, weighed dry, and digested in papainase solution (Worthington) at 60° C. for 16 hours. DNA content was measured using the PicoGreen assay (Invitrogen, Molecular Probes) using Lambda phage DNA as standard. Glycosaminoglycan (GAG) content was quantified using the 1,9-dimethylmethylene blue (DMMB) dye-binding assay with shark chondroitin sulfate as a standard. Total collagen content was determined using acid hydrolysis followed by reaction with p-dimethylaminobenzaldehyde and chloramines. T. Collagen content was estimated by assuming a 1:7.46 hydroxyproline:collagen mass ratio. The interaction index, which is the measured matrix content (DNA, sGAG, or collagen) in the mixed co-culture group normalized by the expected matrix content based on the measured matrix content per in the bAC and hADSC alone groups, was calculated. An interaction index of greater than 1 indicates that the resulting matrix content is higher than expected, while an interactions index of lower than one indicates that the resulting matrix content is lower than expected. An interaction index of one indicates that the resulting matrix content was the same as expected.

Histological Analysis

Cell-hydrogel constructs (n=2) were fixed in 4% paraformaldehyde overnight and stored in 70% ethanol at 4° C. until processed. Constructs were then embedded in paraffin and processed using standard histological procedures. For immunostaining, enzymatic antigen retrieval was performed by incubation in 0.1% Trypsin at 37° C. for 15 minutes. Sections were then blocked with blocking buffer consisting of 2% goat serum, 3% BSA and 0.1% Triton X-100 in 1XPBS, followed by incubation in rabbit polyclonal antibody to collagen type I or II (Abcam) overnight at 4° C. and secondary antibody (Alexa Fluor 488 goat anti-rabbit, Invitrogen) incubation for an hour at room temperature. Nuclei were counterstained with DAPI mounting medium (Vectashield) and images were taken with a Zeiss fluorescence microscope. Sections without primary antibody incubation served as negative controls. A custom image processing program was written in MATLAB (The MathWorks) to quantify the number and size of the cartilage nodules.

Cell Tracking Using Membrane Labeling

To track cell distribution within the hydrogel constructs over time, hADSCS were labeled with red fluorescent dye (PKH26, Sigma) prior to encapsulation at a concentration of 4 μM for 4 minutes following manufacturer's protocol. Labeled hADSCS were co-cultured with bACs in the mixed co-culture hydrogel model at different cell ratios for 21 days (n=3). Samples were fixed in 4% paraformaldehyde overnight, submerged in 30% sucrose solution for 24 hours, embedded in Tissue-Tek (Sakura Finetek), and frozen in liquid nitrogen. Cryosections (12 μm) were washed in DPBS and collagen II and cell nuclei were stained using the immunostaining procedures described above.

Mechanical Testing

Unconfined compression tests were conducted using an Instron 5944 materials testing system (Instron Corporation, Norwood, Mass.) fitted with a 10 N load cell

(Interface Inc., Scottsdale, Ariz.). Cell-hydrogel constructs were tested on day 1 and day 21 of culture (n=4). During testing, cell-hydrogel constructs were submerged in a PBS bath at room temperature. Constructs were compressed at a rate of 1% strain/second to a maximum strain of 15%. Stress versus strain curves were created and curve fit using a third order polynomial equation. The compressive tangent modulus was determined from the curve fit equation at strain values of 15%.

Statistical Analysis

GraphPad Prism (Graphpad Software, San Diego) was used to perform statistical analysis. One- or two-way analysis of variance and pairwise comparisons with Tukey's post-hoc test were used to determined statistical significance (p<0.05). Data was represented as mean±standard deviation of at least three biological replicates.

While the preferred embodiments of the invention have been illustrated and described, it will be appreciated that various changes can be made therein without departing from the spirit and scope of the invention.

What is claimed is:

1. A composition comprising a three-dimensional scaffold encapsulating progenitor cells and tissue-specific primary cells.

2. The composition of claim 1, wherein the progenitor cells are selected from the group consisting of mesenchymal stromal cells, muscle progenitor cells, umbilical cord cells, adult stem cells, and embryonic stem cells.

3. The composition of claim 2, wherein the stem cells are pluripotent stem cells or induced pluripotent stem cells.

4. The composition of claim 1, wherein the number of tissue-specific primary cells is the minimal number needed to promote a therapeutically effective amount of tissue production.

5. The composition of claim 1 comprising a mixed culture of progenitor cells and tissue-specific primary cells.

6. The composition of claim 5, wherein the percentage of primary cells in the mixed culture is 1% or less.

7. The composition of claim 1, wherein the stem cells are adipose-derived stem cells.

8. The composition of claim 1, wherein the tissue-specific primary cells are neonatal articular chondrocytes.

9. The composition of claim 1, comprising adipose-derived stem cells and neonatal articular chondrocytes.

10. The composition of claim 8, wherein cartilage is produced from the composition in an amount effective for treating a subject for a traumatic injury or a disease involving cartilage degeneration.

11. The composition of claim 10, wherein the disease involving cartilage degeneration is arthritis.

12. The composition of claim 9 comprising a mixed culture of adipose-derived stem cells and neonatal articular chondrocytes.

13. The composition of claim 12, wherein the ratio of adipose-derived stem cells to neonatal articular chondrocytes is about 25:75, about 50:50, about 75:25, about 90:10, about 95:5, or about 99:1.

14. The composition of claim 12, wherein the percentage of neonatal articular chondrocytes in the mixed culture is 1% or less.

15. The composition of claim 9 comprising a bilayered culture of adipose-derived stem cells and neonatal articular chondrocytes.

16. The composition of claim 1, wherein the progenitor cells and tissue-specific primary cells are human.

17. The composition of claim 1, wherein the three-dimensional scaffold is a biomimetic scaffold.

18. The composition of claim 1, wherein the three-dimensional scaffold is a hydrogel.

19. The composition of claim 18, wherein the hydrogel binds to paracrine signaling molecules released from the encapsulated cells.

20. The composition of claim 18, wherein the hydrogel comprises chondroitin sulfate methacrylate (CS-MA) and poly(ethylene)glycol diacrylate (PEGDA).

21. The composition of claim 20, wherein the stem cells are adipose-derived stem cells and the tissue-specific primary cells are neonatal articular chondrocytes.

22. The composition of claim 1, further comprising one or more factors selected from the group consisting of a growth factor, an extracellular matrix (ECM) factor, a cytokine, a nutrient, and an antibiotic.

23. The composition of claim 22, wherein the growth factor is selected from the group consisting of a fibroblast growth factor (FGF), an insulin-like growth factor (IGF), and TGF-β.

24. The composition of claim 22, comprising at least one ECM component selected from the group consisting of a proteoglycan, a non-proteoglycan polysaccharide, a fiber, and other ECM component.

25. The composition of claim 24, wherein at least one ECM factor is chondroitin sulfate, heparan sulfate, keratan sulfate, hyaluronic acid, collagen, elastin, fibronectin or laminin.

26. The composition of claim 1, further comprising a pharmaceutically acceptable carrier.

27. A method of treating a subject for tissue damage or loss, the method comprising administering a therapeutically effective amount of the composition of claim 1 to the subject.

28. A method of treating a subject for cartilage damage or loss, the method comprising administering a therapeutically effective amount of the composition of claim 8 to the subject.

29. The method of claim 28, wherein the composition is administered locally at a damaged joint.

30. The method of claim 28, wherein the subject has a traumatic injury or a disease involving cartilage degeneration.

31. The method of claim 30, wherein the disease involving cartilage degeneration is arthritis.

32. A method of generating new tissue in a subject, the method comprising administering the composition of claim 1 to the subject.

33. A method of generating new cartilage in a subject, the method comprising administering the composition of claim 8 to the subject.

34. The method of claim 33, wherein the composition is administered locally to a damaged joint of the subject.

35. The method of claim 33, wherein the subject has a traumatic injury or a disease involving cartilage degeneration.

36. The method of claim 35, wherein the disease involving cartilage degeneration is arthritis.

37. A method of preparing the composition of claim 12, the method comprising:
a) mixing chondroitin sulfate methacrylate (CS-MA) and poly(ethylene)glycol diacrylate (PEGDA) with water;
b) adding adipose-derived stem cells and neonatal articular chondrocytes and media suitable for growth of the adipose-derived stem cells and neonatal articular chondrocytes to form a suspension comprising a mixed culture; and
c) inducing crosslinking of the PEGDA and CSMA to form the hydrogel.

38. The method of claim 37, wherein the ratio of adipose-derived stem cells to neonatal articular chondrocytes is about 25:75, about 50:50, about 75:25, about 90:10, about 95:5, or about 99:1.

39. The method of claim 37, wherein the percentage of neonatal articular chondrocytes in the mixed culture is 1% or less.

40. The method of claim 37, further comprising culturing the adipose-derived stem cells and neonatal articular chondrocytes in the hydrogel under conditions in which the cells proliferate before implantation of the composition in a subject.

41. A method of preparing the composition of claim 15, the method comprising:
a) preparing a first hydrogel encapsulating adipose-derived stem cells;
b) preparing a second hydrogel encapsulating neonatal articular chondrocytes;
c) combining the two hydrogels into a bilayered hydrogel by bringing the first hydrogel and the second hydrogel in contact with each other.

42. The method of claim 41, wherein the first hydrogel and the second hydrogel comprise PEGDA and CSMA.

43. The method of claim 41, further comprising culturing the adipose-derived stem cells and neonatal articular chondrocytes in the hydrogel under conditions in which the cells proliferate before implantation of the composition in a subject.

44. A kit comprising the composition of claim 1 and instructions for treating a traumatic injury or a disease involving tissue degeneration.

45. A kit comprising the composition of claim 12 and instructions for treating a traumatic injury or a disease involving cartilage degeneration.

46. The kit of claim 45, further comprising means for delivering the composition to a subject.

47. A kit comprising the composition of claim 15 and instructions for treating a traumatic injury or a disease involving cartilage degeneration.

48. The kit of claim 47, further comprising means for delivering the composition to a subject.

49. A kit comprising chondroitin sulfate methacrylate (CS-MA), poly(ethylene)glycol diacrylate (PEGDA), adipose-derived stem cells, neonatal articular chondrocytes, and instructions for preparing the composition of claim 12.

50. A kit comprising chondroitin sulfate methacrylate (CS-MA), poly(ethylene)glycol diacrylate (PEGDA), adipose-derived stem cells, neonatal articular chondrocytes, and instructions for preparing the composition of claim 15.

51. A composition comprising a hydrogel encapsulating neonatal articular chondrocytes and conditioned medium, wherein the medium has been conditioned by adipose-derived stem cells.

52. The composition of claim 51, wherein the hydrogel comprises chondroitin sulfate methacrylate (CS-MA) and poly(ethylene)glycol diacrylate (PEGDA).

53. A method of generating new cartilage in a subject, the method comprising administering the composition of claim 51 to the subject.

54. The method of claim 53, wherein the composition is administered locally to a damaged joint of the subject.

55. The method of claim 53, wherein the subject has a traumatic injury or a disease involving cartilage degeneration.

56. The method of claim 55, wherein the disease involving cartilage degeneration is arthritis.

57. A method of preparing the composition of claim 51, the method comprising:
a) mixing chondroitin sulfate methacrylate (CS-MA) and poly(ethylene)glycol diacrylate (PEGDA) with water;
b) adding neonatal articular chondrocytes, media conditioned by adipose-derived stem cells, and media suitable for growth of the neonatal articular chondrocytes to form a suspension; and
c) inducing crosslinking of the PEGDA and CSMA to form the hydrogel.


SEQUENCE LISTING (16 sequences)

SEQ ID NO: 1 - DNA, 20 bp, Artificial Sequence
  human GAPDH forward PCR primer
cgctctctgctcctcctgtt

SEQ ID NO: 2 - DNA, 20 bp, Artificial Sequence
  human GAPDH reverse PCR primer
ccatggtgtctgagcgatgt

SEQ ID NO: 3 - DNA, 20 bp, Artificial Sequence
  bovine GAPDH forward PCR primer
agatggtgaaggtcggagtg

SEQ ID NO: 4 - DNA, 20 bp, Artificial Sequence
  bovine GAPDH reverse PCR primer
gatctcgctcctggaagatg

SEQ ID NO: 5 - DNA, 23 bp, Artificial Sequence
  human aggrecan forward primer
tgaggagggctggaacaagtacc

SEQ ID NO: 6 - DNA, 23 bp, Artificial Sequence
  human aggrecan reverse PCR primer
ggaggtggtaattgcagggaaca

SEQ ID NO: 7 - DNA, 20 bp, Artificial Sequence
  bovine aggrecan forward PCR primer
gtggggcaagactatgatcg

SEQ ID NO: 8 - DNA, 21 bp, Artificial Sequence
  bovine aggrecan reverse PCR primer
gcttgctcctccactaatgtc

SEQ ID NO: 9 - DNA, 20 bp, Artificial Sequence
  human COL2A1 forward PCR primer
tcacgtacactgccctgaag

SEQ ID NO: 10 - DNA, 20 bp, Artificial Sequence
  human COL2A1 reverse PCR primer
ttgcaacggattgtgttgtt

SEQ ID NO: 11 - DNA, 20 bp, Artificial Sequence
  bovine COL2A1 forward PCR primer
tcacgtacactgccctgaag

SEQ ID NO: 12 - DNA, 20 bp, Artificial Sequence
  bovine COL2A1 reverse PCR primer
tgcaatggattgtgttggtt

SEQ ID NO: 13 - DNA, 24 bp, Artificial Sequence
  human COL1A2 forward PCR primer
agggcaacagcaggttcacttaca

SEQ ID NO: 14 - DNA, 24 bp, Artificial Sequence
  human COL1A2 reverse PCR primer
agcgggggaaggagttaatgaaac

SEQ ID NO: 15 - DNA, 20 bp, Artificial Sequence
  bovine COL1A2 forward primer
acattggcccagtctgtttc

SEQ ID NO: 16 - DNA, 20 bp, Artificial Sequence
  bovine COL1A2 reverse PCR primer
gggagggggagtgaattaaa